(19) United States
(12) Patent Application Publication
(10) Pub. No.: US 2008/0009002 A1
(43) Pub. Date: Jan. 10, 2008

(54) Analyte Identification Using Electronic Devices

(75) Inventors: George Gruner, Los Angeles, CA (US); Mikhail Briman, Los Angeles, CA (US)

Correspondence Address: GATES & COOPER LLP;HOWARD HUGHES CENTER, 6701 CENTER DRIVE WEST, SUITE 1050, LOS ANGELES, CA, 90045, US

(73) Assignee: The Regents of the University of California, Aakland (CA)

(21) Appl. No.: 11/667,432

(22) PCT Filed: Nov. 4, 2005
(86) PCT No.: PCT/US05/40295

§ 371 Date: May 9, 2007

Related U.S. Application Data
(60) Provisional application No. 60/626,539, filed on Nov. 9, 2004.

(51) Int. Cl. G01N 27/414; C12Q 1/68; G01N 33/543

(52) U.S. Cl. 435/6; 204/403.01; 204/407; 436/501

(57) ABSTRACT

The disclosure provided herein describes methods and devices for the detection of analytes and/or the characterization of interactions between analytes and compositions capable of binding the analytes. Embodiments of the invention allow the identification and/or characterization of analytes by monitoring the electronic properties of sensors having electronic circuits coupled to compositions capable of binding the analytes under different sensing conditions.

### CROSS REFERENCE TO RELATED APPLICATIONS

This application claims the benefit of U.S. provisional patent application Ser. No. 60/626,539, filed Nov. 9, 2004. This application is related to U.S. patent application Ser. No. 10/431,963 which claims the benefit of U.S. provisional patent application Ser. No. 60/378,843, filed May 8, 2002. The entire content of these patent applications are incorporated herein by reference.

### FIELD OF THE INVENTION

The present invention is directed to optimized methods for the detection, identification and/or quantification of analytes such as polynucleotides and polypeptides as well as systems and devices adapted for performing these methods.

### BACKGROUND OF THE INVENTION

Label free detection of the presence of and binding between biomolecules such as analytes and compositions capable of binding such molecules, coupled with a high sensitivity offer enormous advantages in sensing applications. These methods can for example exploit the fact that different biomolecules (such as a full complementary and single base mismatch in case of DNA duplex formation) exhibit different binding strength when a binding to a device or between biomolecules, such as DNA duplex formation or ligand-receptor binding occurs.

Electronic devices such as resistors, and field effect transistors with nanowire conducting channels have been employed in the past for bio-sensing applications. A variety of Field Effect Transistor (FET) configurations, where the conducting channel is in direct contact with the environment have been employed for biosensing and monitoring applications, the detection occurring in biological buffer or after buffer removal Electronic detection in buffer environments has been demonstrated (see, e.g. R. J. Chen et al PNAS 100, 4989 (2003), Li et al Nano Letters 4, 245 (2004), J Hahm and C Lieber Nano Letters 4 (2004)). Detection after the buffer removal has also been used, with the advantage of a simpler detection scheme and the avoidance of complications resulting in the conducting buffer.

The interaction between the bio-molecules such as DNA duplex formation, ligand receptor binding can lead to changes of the electronic characteristics of a device. In such interactions described in the art, only the source-drain current at fixed gate voltage or no gate applied, and thus only the resistance change is measured (see, e.g. Li et al Nano Lett 4, 245 (2004), J Hahm and C. Lieber Nano Letters 4, 51 (2004)). In a few cases the detection of the transistor characteristics have also been reported (see, e.g. R. J. Chen et al PNAS 100, 4989 (2003), K. Bradley et al Nano Letters 4, 253 (2004)).

The specificity of detection is a major issue for analyte identification. One avenue used extensively is the application of a layer that avoids nonspecific binding, such as a PEG and PEG/PEI layer, together with the deposition of a probe biomolecule that interacts with the target molecule. Such scheme has been described by Star et al Nano Letters 3, 459(2003). Methods described in the art do not distinguish between binding events involving the target molecule or another biomolecule that also may bind (with a different binding strength) to a probe, or to a surface of a device.

The experiments described in the literature using electronic devices have been conducted at constant temperature and pH conditions. Due to this limitation, the methods described in the art cannot for example discriminate between different oligomers if their concentrations are not known. Methods where the temperature was varied have employed only optical methods (H. Urakawa et al Applied and Env. Microbiology 69, 2848 (2003). In general these hybridization conditions (temperature, ionic strength) and washing conditions (dissociation) are used together with optical readout (see, e.g. Zheng et al Appl. Environ. Microbiol. 62, 4504 (1996)). However, these methods require labeling, a factor that may influence and therefore confound DNA melting curves, protein denaturation, and ligand-receptor binding analyses.

### SUMMARY OF THE INVENTION

The disclosure provided herein describes methods, devices and/or systems for the detection of analytes and/or the characterization of interactions between analytes and compositions capable of binding the analytes. These methods and devices have a number of embodiments. One illustrative embodiment of the invention is a method of sensing binding of an analyte to a composition capable of binding the analyte by exposing the analyte to a sensor that includes an electric circuit coupleable to a device that monitors alterations in an electrical property of the electric circuit and a composition capable of binding the analyte coupled to the electric circuit; wherein an electrical property of the electric circuit is altered when the analyte binds the composition capable of binding the analyte. In this method, a user can monitor an electrical property of the sensor under a first sensing condition comprising a first temperature, a first pH, a first buffer composition or a first electric field and subsequently monitor an electrical property of the sensor under a second sensing condition comprising a second temperature, a second pH, a second buffer composition or a second electric field. In this embodiment of the invention, a change in an electrical property under the first sensing condition as compared to the second sensing condition correlates to the binding of the analyte to the composition capable of binding the analyte. Consequently, in this method one can sense the binding of the analyte to the composition capable of binding the analyte by observing a change in the electrical property observed under the first sensing condition as compared to the second sensing condition. Typically, the electrical property of the electrical circuit that is monitored is current, voltage or capacitance.

A wide variety of compositions capable of binding analytes are known in the art that can be used with embodiments of the invention. In certain embodiments of the invention, the composition capable of binding the analyte comprises a polypeptide such as an antibody or a cellular receptor. Alternatively, the composition capable of binding the analyte comprises a polynucleotide such as a single stranded DNA molecule. In a specific embodiment of the invention, the analyte comprises a target polynucleotide and the composition capable of binding the analyte comprises a sensing polynucleotide having sequences complementary to the target polynucleotide. In certain embodiments of the invention, the change in the electrical property observed under the first sensing condition as compared to the second sensing condition is correlated to the degree of complementarity between the target polynucleotide and the polynucleotide having sequences complementary to the test polynucleotide.

A related embodiment of the invention is an analyte sensor system comprising an electric circuit coupleable to a device that monitors alterations in an electrical property of the electric circuit, a composition capable of binding the analyte coupled to the electric circuit; wherein an electrical property of the electric circuit is altered when the analyte binds the composition capable of binding the analyte, a fluid delivery element adapted to deliver a fluid that may contain an analyte to the sensor, a sensor conditioning element adapted to alter the condition under which the analyte is sensed so that the system is adapted to monitor an electrical property of the sensor under a first sensing condition comprising a first temperature, a first pH, a first buffer composition or a first electric field; and subsequently monitor an electrical property of the sensor under a second sensing condition comprising a second temperature, a second pH, a second buffer composition or a second electric field. In this embodiment, the system is arranged so that a change in an electrical property under the first sensing condition as compared to the second sensing condition correlates to the binding of the analyte to the composition capable of binding the analyte. Optionally, the system comprises a plurality of sensors to perform the method on a plurality of different analytes as part of a sensing array and/or one or more sensors having a plurality of compositions capable of binding a plurality of different analytes to sense a plurality of different analytes.

In certain embodiments of the invention, the system comprises a resistor, a transistor or a capacitor. In some embodiments of the invention, the system is coupled to a processor that compares the electric property observed under the first sensing condition and the electrical property observed under the second sensing condition. In some embodiments, the processor uses an algorithm to provide a signal when the difference between the electric property observed under the first sensing condition and the electrical property observed under the second sensing condition meets a predefined parameter.

The invention also provides additional articles of manufacture including sensor elements, sensor sets and kits. In one such embodiment of the invention, a kit and/or sensor element or set, useful for the sensing an analyte as is described above, is provided. The kit and/or sensor set typically comprises a container and a sensor as described above. A typical embodiment is a kit comprising a container and, within the container, an analyte sensor apparatus having a design as disclosed herein and instructions for using the analyte sensor apparatus.

### BRIEF DESCRIPTION OF THE FIGURES

FIG. 1A shows the schematic outlay of the semiconductor sensor. An applied voltage leads to a current which is monitored. FIG. 1B shows semiconductor nanoparticles with their surface layer. The arrow indicates the electronic conduction path. FIG. 1C shows a semiconductor nanoparticle coated with another material, with the arrows indicating the electronic conduction path.

FIG. 2 is a schematic representation of the functionalized nano-structured sensor-material, with a target bio-material also shown.

FIG. 3 provides a schematic of the nanotube field effect transistor (NTFET) that uses a network of nanoparticles as conducting channel. A polymeric functional layer, which coats the network, functionalized with a molecular receptor, a protein that recognizes a biomolecule (not shown) can be incorporated into the structure. S; source, D; drain, G: gate. (Dimensions are not in scale.)

FIG. 4 provides an illustration of a biotinylation reaction of the polymer layer (PEI and PEG).

FIG. 5 provides an AFM image of the polymer-coated and biotinylated NTFET after exposure to streptavidin labeled with gold nanoparticles (10 nm diameter).

FIG. 6 provides gate voltage dependence of the source-drain current Isd of a typical device before and after PEI/PEG polymer coating and after biotin attachment to the polymer layer.

FIG. 7 provides change of the device characteristic Isd(Vg) upon exposure to streptavidin. Different devices with similar characteristic as observed before polymer coating were used for a, b, and c. (a) Gate voltage dependence Isd of the biotinylated, polymer-coated NTFET in the absence and in the presence of streptavidin. (b) Current-voltage dependence Isd(Vg) of the bare NTFET device to nonspecific protein binding. (c) Isd(Vg) of the polymer-coated NTFET device in the absence and presence of streptavidin. (d) Isd(Vg) of the biotinylated, polymer coated NTFET device in the absence and presence of streptavidin that was preincubated with biotin.

FIG. 8 provides a graph of data in experiments examining the affinity of proteins to nanotubes using multiwall nanotubes. Nanotubes (2 mg) dispersed in were incubated with BSA-dye conjugate (1 mg/ml, 1.25 microgram) overnight, were washed with PBS. Depletion of BSA from the solution was evaluated using UV absorption (580 nm) of fluorescent dye. As shown in this graph, when adding proteins (BSA) the resistance drops. This is due to a combination of factors: due to the conducting channel provided by the buffer and due to the change of the resistance of the network due to protein attachment. When appropriate correction for the buffer resistance is made, the resulting network resistance (considering the film to be in parallel with buffer) it is approximately 30% higher than the value of the resistance in just buffer. This graph also illustrates the resistance of network device in air, and after application of buffer and BSA. It is apparent that the network resistance measured after buffer removal is higher than before the protein attachment.

FIG. 9 provides a graph of data from a simple analysis made where the values of the network's resistance are assumed to correspond to the values measured on the “wet” section of the graph.

FIGS. 10A-10D provide a graph of data from experiments as described in Example 2 below showing film resistance under various conditions with only buffer or buffer+protein is applied.

FIG. 11 shows the electric field dependence of the change of the device characteristics upon incubation with streptavidin. The device was incubated with streptavidin and placed in 150 mM PBS buffer. The 40 mV shift to the right of the black curve indicates that more negative average electric field (Vg offset) leads to less charge transfer from adsorbed proteins.

FIG. 12 shows the pH sweep dependence of the device characteristics for two proteins. The shift of each device is taken relative to the pH response of the device before incubation. FIG. 12A: pH response of bare and SA covered device. FIG. 12B: pH response of bare and BSA covered device. FIG. 12C: Relative I_{sd}-V_{g} shifts of SA and BSA covered devices.

FIG. 13 shows the electrical dependence of the device. The device was placed in 150 mM phosphate buffered saline (PBS), pH 7.4, T=25 C, and biased with Vsd=20 mV.

FIG. 14 shows the use of the technique to monitor nonspecific binding in real time. The stable device in 150 mM PBS, pH 7.4, T=25 C was exposed to 10 nM Streptavidin. Black curve in the insert is the first I_{sd}-V_{g} curve at t=0 (s), and red one is the final measurement at t=30,000 (s). The parasitic currents (Ig) were below one nano ampere at all times. Rinse at t=28,000 (s) didn't change the signal, indicating that streptavidin is irreversibly bound to nanotubes.

FIG. 15 shows the schematics of a typical experimental setup. Temperature of the buffer can be changed from 20 C to 100 C. pH is varied by addition of HCl or NaOH with the same ionic strength as buffer.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

Interactions between biological molecules, such as polynucleotides (e.g. DNAs and mRNAs), polypeptides (e.g. proteins) and larger structures (e.g. viral coat protein complexes and/or viruses), together with the conformations of biomolecules depend on a number of conditions under which the interaction occurs including temperature, pH and buffer composition. In addition, biomolecules are charged entities, and electrostatic interactions, and electric fields influence the binding strength. Various biomolecules undergo changes when conditions such as the temperature or pH is varied. These changes, and the temperature where they occur represent the binding of the biomolecules, such as the binding between the two complementary DNA strands. This binding is different for full complementary and for oligomers that differ by one or more base pair sequence, such as in the case of SNIPs. Thus, measurement of the device characteristics as the function of this external parameter offers the identification of the differences between DNA segments. Similar effects occur when the pH of the buffer is varied, and thus the variation of this parameter also offers an opportunity for detection of differences between the different base pair sequences. In addition, an applied electric field can also influence and modulate duplex formation or alternatively unbinding, of analytes such as polynucleotides and polypeptides.

Embodiments of the invention described below, allow the identification and/or characterization of various analytes through the measurement of electronic properties of devices having electronic circuits where parameters that modulate the properties of the analytes and their binding features are varied. In particular embodiments, the invention exploits the utilization of the change of various parameters such as temperature (T), pH and bottom gate (Vg) as the means of identification of analytes, together with the variation of the ionic strength and other solvents, such as formaldehyde in case of DNA detection.

One illustrative embodiment of the invention is a method of sensing binding of an analyte to a composition capable of binding the analyte by exposing a fluid containing the analyte to a sensor comprising an electric circuit coupleable to a device that monitors alterations in an electrical property of the electric circuit and a composition capable of binding the analyte coupled to the electric circuit; wherein an electrical property of the electric circuit is altered when the analyte binds the composition capable of binding the analyte. In this embodiment, the sensor is then monitored under a first sensing condition comprising a first temperature, a first pH, a first buffer composition or a first electric field and subsequently monitored under a second sensing condition comprising a second temperature, a second pH, a second buffer composition or a second electric field. In this embodiment, a change in an electrical property under the first sensing condition as compared to the second sensing condition correlates to the binding of the analyte to the composition capable of binding the analyte. Consequently one can sense the binding of the analyte to the composition capable of binding the analyte by observing a change in the electrical property observed under the first sensing condition as compared to the second sensing condition. Optionally, the system is adapted to continuously monitor an electrical property of the electrical circuit under different sensing conditions. Typically, the electrical property of the electrical circuit that is monitored is current, voltage (AC or DC) or capacitance, properties which encompass related properties such as inductance, resistance, impedance, conductance, transconductance etc.

As the interaction between analytes and compositions capable of binding analytes is controllable by altering binding conditions including temperature, pH, buffer composition or electric fields, in certain embodiments of the invention, one or more sensing conditions is altered so as to alter the binding of the analyte to the composition capable of binding the analyte. In certain methods of the invention, the first and second sensing condition that is monitored comprises the same parameter (e.g. a first and second temperature, a first and second pH, a first and second electric field etc.). In alternative embodiments, wherein the first and second sensing conditions different parameter (e.g. a first temperature and a second pH, a first temperature and second electric field etc.). Optionally, multiple sensing conditions are manipulated. In an illustrative embodiment, the first and second sensing conditions are a first and second electric field. Alternatively, the first and second sensing conditions are a first and second pH, a first and second buffer condition or a first and second temperature.

A huge number of compositions capable of binding a wide variety of different types of analytes are known in the art. Optionally, the composition capable of binding the analyte comprises a polypeptide. In one embodiment, the analyte comprises an antigen and the composition capable of binding the analyte comprises an antibody that binds the antigen. Alternatively, the analyte can comprise an antibody and the composition capable of binding the analyte can comprise an antigen bound by the antibody. In another embodiment of the invention, the analyte can comprises a polypeptide receptor and the composition capable of binding the analyte comprises a ligand bound by the polypeptide receptor. Alternatively, the analyte can comprise a polypeptide ligand that can be bound by a polypeptide receptor and the composition capable of binding the analyte comprises the polypeptide receptor.

In some embodiments of the invention, the composition capable of sensing an analyte comprises a polynucleotide, for example a polynucleotide known in the art to bind a specific polynucleotide or polypeptide sequence. In a specific embodiment of the invention, the analyte comprises a target polynucleotide (i.e. the target of the sensing device) and the composition capable of binding the analyte comprises a homologous sensing polynucleotide having sequences complementary to the target polynucleotide. In certain embodiments, the change in the electrical property observed under the first sensing condition as compared to the second sensing condition can be correlated to the degree of complementarity, for example the relative (or total) number of complimentary base pairs shared between the target polynucleotide and the polynucleotide having sequences complementary to the test polynucleotide.

In other embodiments of the invention, the composition capable of binding the analyte is not a polypeptide or polynucleotide and instead is a chemical compound(s) known to be specific for the analyte(s) of interest. In some embodiments of the invention, the change in the electrical property observed under the first sensing condition as compared to the second sensing condition is correlated to the degree of affinity or avidity between the analyte and the composition capable of binding the analyte. In certain embodiments of the invention, a sensing condition such as the electric field, pH, temperature or buffer condition is altered so as to alter the binding of the analyte to the composition capable of binding the analyte.

The methods and devices disclosed herein can be adapted for use in a wide variety of contexts. For example, the method or device can use a plurality of sensors to perform the method on a plurality of different analytes as part of a sensing array. Alternatively, the method or device can use a single sensor that comprises a plurality of compositions capable of binding a plurality of different analytes to sense a plurality of different analytes.

Yet another embodiment of the invention is an analyte sensor device and/or system for practicing a method of the invention comprising an electric circuit coupleable to a device that monitors alterations in an electrical property of the electric circuit, a composition capable of binding the analyte coupled to the electric circuit; wherein an electrical property of the electric circuit is altered when the analyte binds the composition capable of binding the analyte, a fluid delivery element adapted to deliver a fluid that may contain an analyte to the sensor, and a sensor conditioning element adapted to alter the condition under which the analyte is sensed so that the system is adapted to monitor an electrical property of the sensor under a first sensing condition comprising a first temperature, a first pH, a first buffer composition or a first electric field; and subsequently monitor an electrical property of the sensor under a second sensing condition comprising a second temperature, a second pH, a second buffer composition or a second electric field. In this system, a change in an electrical property under the first sensing condition as compared to the second sensing condition correlates to the binding of the analyte to the composition capable of binding the analyte.

Optionally, the device or system is coupleable to a processor (e.g. a computer or the like) that compares the electric property observed under the first sensing condition and the electrical property observed under the second sensing condition. In certain embodiments of the invention, the processor uses an algorithm to provide a signal when the difference between the electric property observed under the first sensing condition and the electrical property observed under the second sensing condition meets a predefined parameter. Optionally, this signal is visible (e.g. a red light) or auditory (e.g. a chime). In some embodiments of the invention, the system or device comprises a resistor, a transistor or a capacitor. In some embodiments of the invention, the sensor comprises an insulating layer that inhibits binding of an analyte to a portion of the sensor. Optionally, the fluid delivery element of the system is adapted to circulate a fluid over the sensor. The fluid delivery element can comprise any one of a variety of such elements known in the art and typically comprises a fluid pump, a fluid conduit or a pipette. The sensor conditioning element can comprise a wide variety of such elements that are used in the art to modulate the conditions under which molecules interact. In certain embodiments of the invention, the sensor conditioning element comprises a heater, a mixer (e.g. a stir bar etc.), a fluid conduit (e.g. tubing) or a pipette (e.g. one that delivers a fluid such as a acidic or basic solution designed to alter pH). Optionally the system further comprises a device that allows one to monitor the sensing condition, for example a thermometer, a pH meter or the like.

In some embodiments of the invention, a change in the electrical property observed under the first sensing condition as compared to the second sensing condition is correlated to affinity or avidity that the analyte has for the composition capable of binding the analyte. In practicing the analyte or the composition capable of binding the analyte is typically not labelled with a detectable marker. In some embodiments of the invention, the sensor comprises a nanostructured material (e.g. a conducting channel constructed from a nanomaterial such as a nanoparticle network). Alternatively, the sensor does not comprise a nanostructured material. The system can comprise a plurality of sensors to perform the method on a plurality of different analytes as part of a sensing array; and/or a plurality of compositions capable of binding a plurality of different analytes to sense a plurality of different analytes.

The disclosure provided herein also describes methods and device for the electronic monitoring of analytes such as biomolecules (i.e. those molecules derived from a living organism) under different conditions in order to observe conformational changes and/or binding interactions. As noted above, certain embodiments of the methods disclosed herein can employ semiconductor nanostructured materials such as nano-structured chemical sensors (see, e.g. Solid State Gas Sensors Eds. P. T. Moseley and B. C. Tofield (Adam Hilger, Bristol 1987)). Typical nano-structured sensors are sensors where the sensing element comprises a material that exhibits (e.g. one manufactured to exhibit) a desirable structural characteristic on the length scale of less that 100 nm (e.g. nanotubes and the like). An illustrative example of such sensing elements are nanoparticles of an oxide semiconductor or a non-porous material. Another illustrative example is a nanostructured material coated with a layer sensitive to an analyte. The structural attributes of such sensing elements leads to a large surface area which is available for immobilization of a biomaterial which then facilitates their use in methods for the electronic monitoring of biomolecules. The detection of sensing involves the transfer of a signal between two nanostructured materials in contact with each other, this signal can be electronic or mechanical. In this context, elements such as nano-structured chemical sensors can be used as biosensors by exploiting their surface layer as a medium for attachment of biomolecules as well as for the evaluation of the status of these biomolecules by monitoring the resistance across the material.

Another embodiment of the invention is a method for detecting an interaction between a putative binding partner and a biomolecule coupled to a material having measurable level of resistance (e.g. a nanostructured semiconductor material), the method comprising measuring the level of resistance of semiconductor nanostructured material coupled to the biomolecule under one or more sensing conditions, for example in the presence of a medium without a putative binding partner; and then exposing the semiconductor nanostructured material coupled to the biomolecule to a medium having a putative a binding partner; measuring the level of resistance of semiconductor nanostructured material coupled to the biomolecule in the presence of a medium having a putative a binding partner; wherein the level of resistance of so measured is modulated by the binding status of biomolecule such that a change in the level of resistance so measured provides an indication of an interaction between the binding partner and the biomolecule. Certain embodiments of this method analyzes these interactions under a first and second sensing condition as described above.

Another embodiment of the invention is a method for detecting a conformational change in a biomolecule coupled to a semiconductor nanostructured material having measurable level of resistance, the method comprising measuring the level of resistance of semiconductor nanostructured material coupled to the biomolecule in a first conformation, exposing the semiconductor nanostructured material coupled to the biomolecule to a medium having molecules which may induce a conformational change in the biomolecule, measuring the level of resistance of semiconductor nanostructured material coupled to the biomolecule in the presence of the medium having molecules which may induce a conformational change in the biomolecule, wherein the level of resistance of so measured is modulated by the conformational state of biomolecules such that a change in the level of resistance so measured provides an indication of a change in the conformation of the biomolecule. Certain embodiments of this method analyzes these interactions under a first and second sensing condition as described above.

Another embodiment of the system or device is a nano-structure biosensor comprising a source electrode, a drain electrode, a nanoparticle network (optionally having a polymer layer such as poly(ethylene glycol) (EG) or polyethylene imine (PE) disposed upon it in order to inhibit nonspecific binding of the molecule to be sensed) connecting the source electrode and the drain electrode, a gate electrode; and a polynucleotide (e.g. a single stranded DNA oligonucleotide) or polypeptide (e.g. antibody) capable of specifically binding a molecule to be sensed, wherein the polynucleotide or polypeptide capable of specifically binding a molecule to be sensed is coupled to the nanoparticle network. Certain embodiments of this device analyze these binding interactions under a first and second sensing condition as described above.

Optionally in such embodiments, the polynucleotide or polypeptide capable of specifically binding a molecule to be sensed is disposed within the polymer layer. In certain embodiments of the invention, the nanoparticle network comprises a plurality of nanotubes, a plurality of nanofibres, a plurality of nanowires, a plurality of nanococoons or a plurality of semiconducting nanoparticles. In some embodiments of the invention, the device comprises comprising one or more electronic leads coupled to the network, for example one or more electronic leads are in an electronic lead arrangement that compensates for buffer conductance.

Yet another embodiment of the invention is a method for detecting an interaction between a polypeptide or a polynucleotide and a binding partner for the polypeptide or the polynucleotide comprising measuring the level of resistance between a source electrode and a drain electrode in a nano-structure biosensor comprising the source electrode, the drain electrode, a nanoparticle network connecting the source electrode and the drain electrode, a gate electrode; and a polynucleotide or polypeptide capable of specifically binding a molecule to be sensed coupled to the nanoparticle network, wherein level of resistance between the source electrode and the drain electrode is correlated to the presence of an interaction between the polypeptide or the polynucleotide and binding partner for the polypeptide or the polynucleotide, so that the interaction between the polypeptide or the polynucleotide and the binding partner for the polypeptide or the polynucleotide is detected. Certain embodiments of this device analyze these binding interactions under a first and second sensing condition as described above. Optionally, the interaction between the polypeptide or the polynucleotide and the binding partner for the polypeptide or the polynucleotide is detected in a buffered aqueous solution.

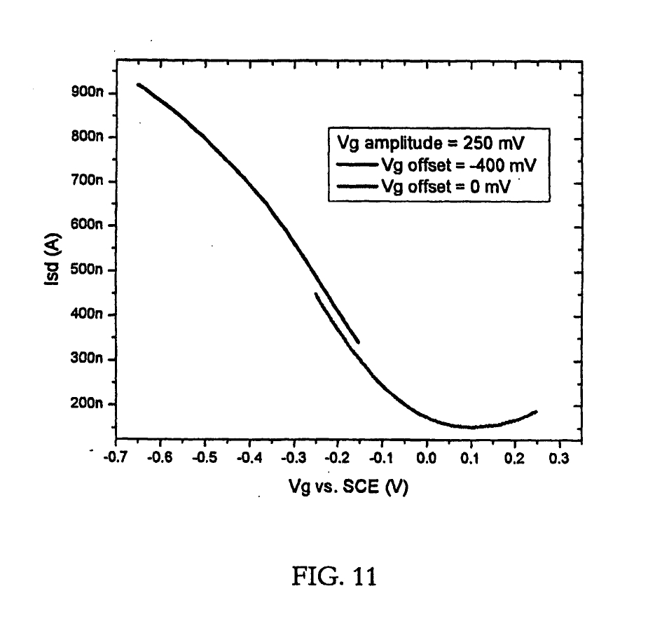

As disclosed herein (see e.g. Example 3 and FIG. 11), method and device characteristics, as can be monitored in a buffer environment using so-called liquid gating can depend on the application of a voltage between the surface of the device and a conducting film that is insulated from the device used for the monitoring of the biological process. In both cases there is an insulating layer between the surface onto which the biomolecule is immobilized, and another conducting layer that is not in contact with the buffer. Depending on the polarity of the voltage that is applied between the device and the conducting layer, different device characteristics, indicating a different amount or different configuration of the biomolecule. Electric field thus can influence the binding of biomolecules to the surfaces of the device and consequently can also be used to un-bind the species from the surface, or from an immobilized biomolecule to which they are bound.

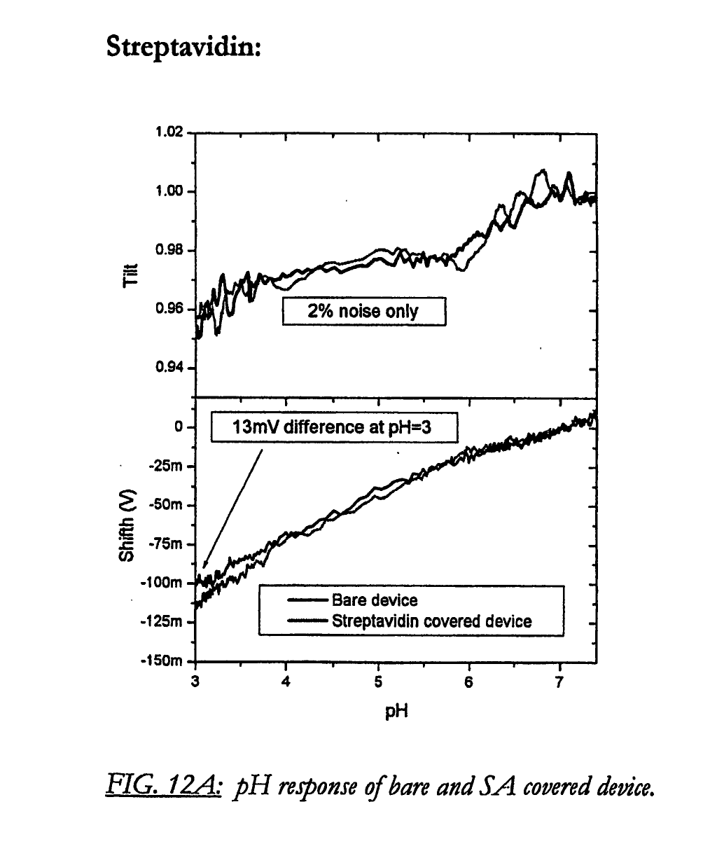

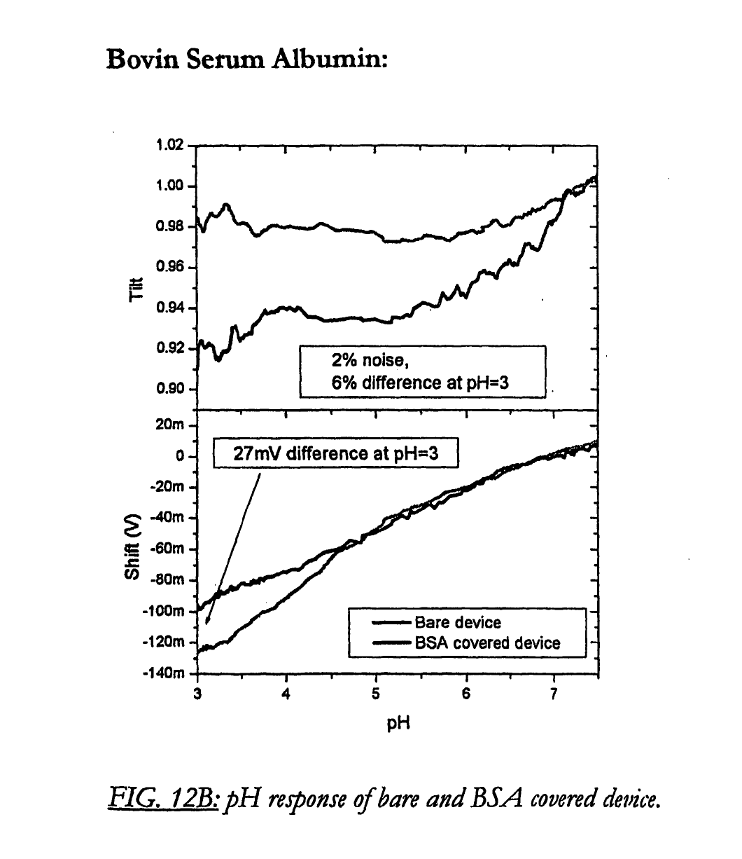

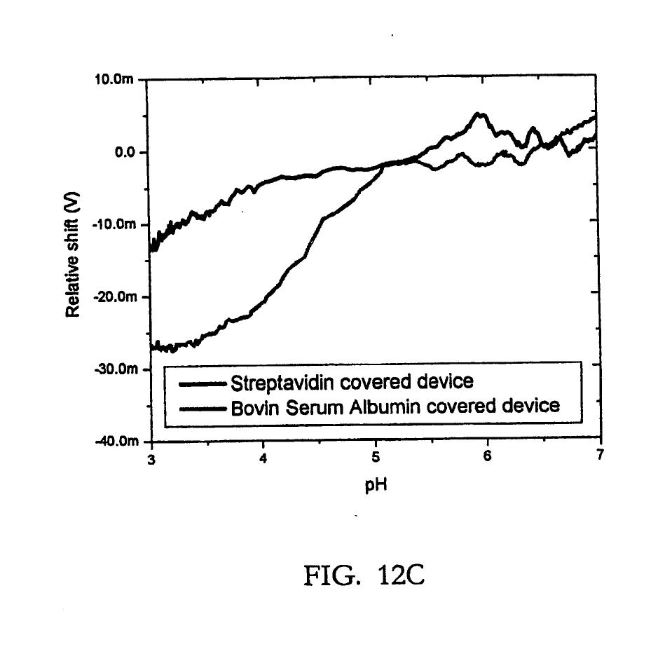

Conformational states (such as the various forms of DNA, proteins—and interaction between biomolecules) such as the melting temperature of DNA, and denaturing temperatures of ONA and proteins—also depends on factors such as the pH and temperature. In turn the interaction between biomolecules and the devices are also pH dependent, allowing the identification of biomolecules and interactions to be identified by varying the pH and monitoring the change of the device characteristics. Such identification, using streptavidin and albumin as examples are described in Example 3 and is displayed in FIG. 12.

The effect of the temperature is similar to the effect of the change of pH. In particular, both parameters can influence DNA duplex formation and also the conformational state of proteins and other biomolecules. Thus temperature can be used as an external parameter to identify biomolecules.

The methods discussed above can also be used for arrays of biosensors where each electronic device element is functionalized individually for different biomolecules, and the electronic response of each element is monitored as one, or all of the parameters, the temperature, pH, solvent, and electric field are varied. The method can be applied also for keeping these parameters constant (but different form ambient temperature, physiological buffet pH or zero electric field) and performing a statistical analysis of the responses of the individual devices that form the array. In this case the analysis is similar to that used for the optical response of the various spots in gene chips for example. One such measurement method that allows detection and monitoring in physiological buffer is described in Example 3.

The invention disclosed herein can be used with nano-structured sensors where the detection involves two nanostructured materials in contact, with the detection involving the transmission if a signal between the two nano-structured elements. A typical sensor can be a chemical sensor (see, e.g. Solid State Gas Sensors Eds. P. T. Moseley and B. C. Tofield (Adam Hilger, Bristol 1987)), or as a biosensor by exploiting the surface layer both as a medium for attachment of biomolecules and detection through the resistance across the material, or across another material in contact with the surface layer. Typical nano-structured sensors are sensors where the sensing element consists of a material where at least one of the architectural features of the material is selected for use in the invention in part because it has length of less that 100 nm. For example, a “nanowire” is defined as a material (wire, fiber, cone or tube) with at least one dimension less than 100 nm. One representative example of such a sensing element are nano-particles of an oxide semiconductor or a non-porous material This structural attribute of this sensing element leads to a large surface area which is available for immobilization of a biomaterial. Consequently, such semiconductor nanostructured materials can be utilized for the electronic monitoring of biomolecules which undergo conformational changes and/or ligand binding. Another example is a nanostructured material on which another material, such as a polymer film or lipid bilayer is deposited.

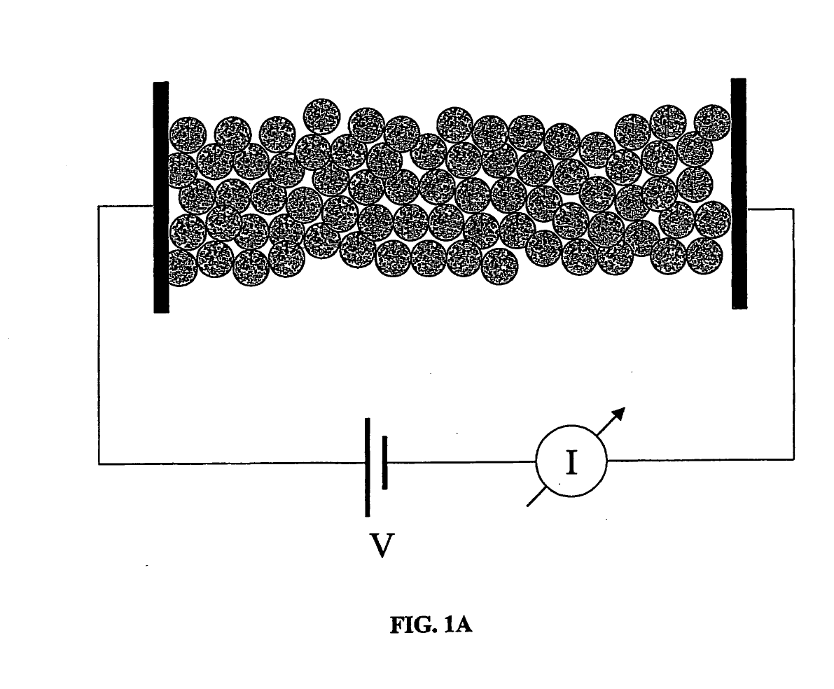

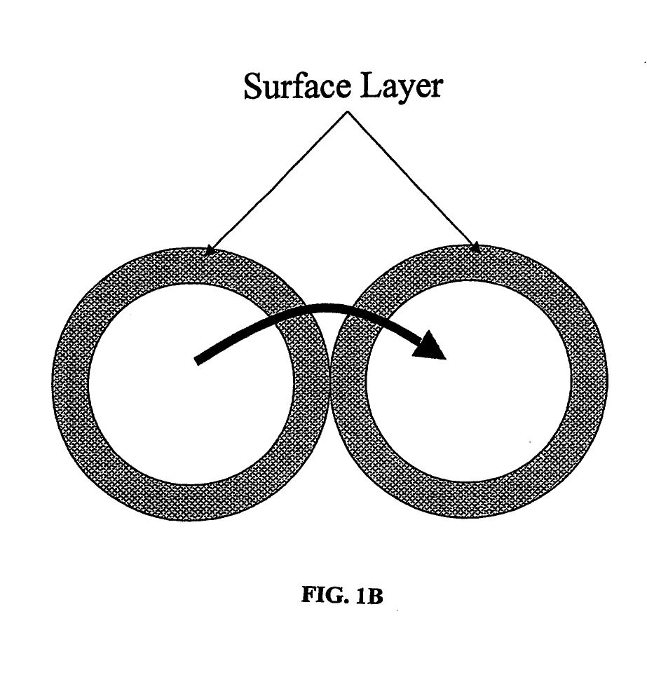

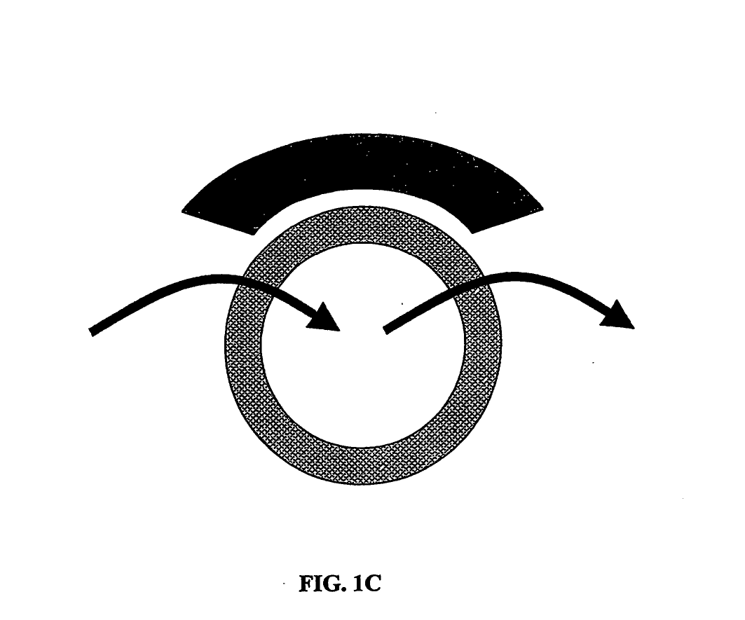

Such materials have been employed earlier as a chemical sensor. Among the various semiconductor nanostructured sensors oxide semiconductors, such as SnO₂ are the most widely used nano-porous material used for sensing of chemical elements (see, e.g. S. Trautweiler et al.; New Silicon based metal-Oxide Chemical sensors www.sensormag.com/articles/0999). The sensing is through the resistance, which changes upon the adsorption of the species on the material. The operation of the oxide semiconductor based chemical sensor is as follows. The oxide semiconductor is n-type, having an excess electrons, this achieved by doping. Due to the n-type character of the carriers, oxygen molecules adsorbed at the surface dissociate creating O (minus) species and thus an equilibrium charged surface layer, which in general is called the depletion layer. The surface of the material can also be functionalized by depositing an appropriate material on the surface, such functionalization routes are well established for various oxide materials. Other species, such as DNA oligomers or proteins which may attach to the immobilized biomaterial lead to changes in the overall electron concentration at the surface—which is detected through the change of the resistance measured across the (semiconducting) SnO₂ film, deposited onto an appropriate surface. Extraordinary detection sensitivity can be achieved, because the transport of electronic charges across the surface layers is extremely sensitive to charge or dipole induced modifications of the layer. The device is displayed in FIG. 1A, with the structural elements—nano-particles across which electronic conduction occurs—displayed on FIG. 1B. The arrow indicates the path of electronic conduction across the depletion layer. In another embodiment, a layer, sensitive to the analyte can be deposited on the nanostructured material, and transduction of a signal from the deposited material to the nanostructured material leads to the change of the resistance of the nanostructured material.

The detection of conformational changes, interaction between proteins and DNA duplex formation is achieved as follows. On the surface of the sensor a biomolecule (the probe) is deposited and immobilized using well know techniques for biomaterial immobilization. Depending on the use, a typical biomaterial can be a molecules such as a: a single stranded DNA oligonucleotide; an RNA; an antibody to the protein one intends to detect; and any one of a wide variety of polynucleotides and polypeptides known to interact with binding partner such as a complimentary polynucleotide, a ligand (including small molecules) etc. In this context, the introduction of complimentary DNA, or antigen which binds to the antibody or polypeptides that interact with etc., the binding partner changes of the surface characteristics leads to a change of an electronic signal between the nano-particles, which is detected. Artisans understand that devices, methods and materials of the invention can be used to examine the interaction of both biomolecules as well as analogously interacting chemical species.

Typically, the biomolecule detected by the methods and devices of the invention is an analyte. The term “analyte” is used according to its broad definition of a substance being identified and/or measured in an analysis and includes without limitation any substance or chemical constituent of a fluid such as a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensing regions, devices, and methods is a polypeptide such as an antibody or a polynucleotide such as mRNAs and DNAs. Salts, sugars, proteins, fats, vitamins and hormones naturally occurring in blood or interstitial fluids can constitute analytes in certain embodiments of the invention. The analyte can be naturally present in the biological fluid or endogenous, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin. The metabolic products of drugs and pharmaceutical compositions are also contemplated analytes.

A typical configuration in embodiments of the invention includes the nanostructured material on which another material is deposited. For example, biomolecules can be immobilized on the surface of the deposited material. Conformational changes or ligand binding can then be detected by transducing an electronic or mechanical signal—that arises as the consequence of the conformational change, duplex formation, antibody-antigen or ligand binding—in the deposited material to the nanostructured material. Other materials include polymers that prevent non-specific bio-molecule binding and a layer of molecules on which bio-molecules are attached.

Other embodiments of the invention include a nanostructured material network on a substrate. Preferable network properties include an interconnected network so that the current (or most of the current) flows across the network. Preferable network properties further include a “loose” network so that the elements of the network in contact with the environment. Network density optimization can be important and is probably dose to a “percolation threshold”. Preferable elements for use with embodiments of the invention include nanowires, nanotubes, and nanoparticles, such as metal oxides. Preferable elements for use with embodiments of the invention further include electronic leads to the network (e.g. a gate arrangement for transistor operation).

Typical embodiments of the invention can also include a layer for bio-functionality, typically recognition molecules (e.g. single strand oligomer, antibody etc.) attached to the network, a coating to prevent false positives, a coating including recognition molecules and/or a coating to which recognition molecules, antibodies are attached (e.g. an electronic lead arrangement for buffer conductance compensation).

In certain embodiments of the invention include compensation for buffer conductivity (e.g. same arrangement as above but without the nanoparticle network).

In certain embodiments of the invention, detection methods include: dc voltage or current; ac voltage and current; electrical pulses; and/or electrical fluctuations due to the interactions between biomolecules (e.g. proteins/DNA). Embodiments of the invention include bio-molecule detection: (1) in buffer, e.g. for real time monitoring the change of resistance; and/or (2) after buffer removal by detecting the attachment or presence of bio-molecule by measuring the change of resistance with and without the target molecule.

In some embodiments of the invention a first step in the functionalization of the surface is to attach a molecule or reactive group which provides a common link for a number of subsequent, different, specific functionalizations. For example, biotin can be coupled to semiconductor surfaces through known methods (see, e.g. A. N. Asanov et al, Anal. Chem. 70, 1156 (1998). In such embodiments, after, biotinylated oligonucleotides, biotinylated secondary antibodies, etc., can be coupled through a streptavidin bridge. Alternatively, probe molecules, e.g. DNA oligonucleotides, can be directly adsorbed on the surface (see, e.g. P. M. Armistead and H. Holden Thorp, Anal. Chem. 73, 558 (2001); adsorbing a secondary antibody would provide binding sites to any specific primary antibody. By electronically detecting ligand binding, an array of such μm scale sensors can allow thousands of binding assays to be run in parallel with very small quantities of material.

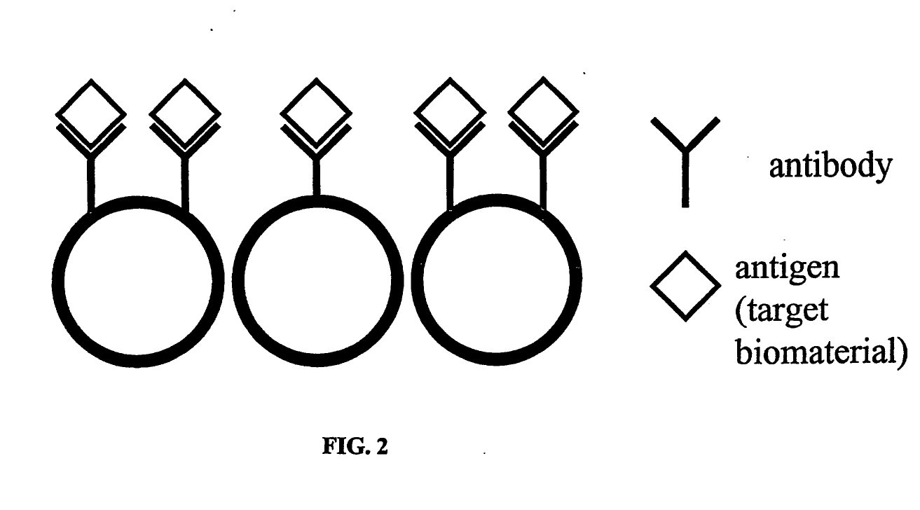

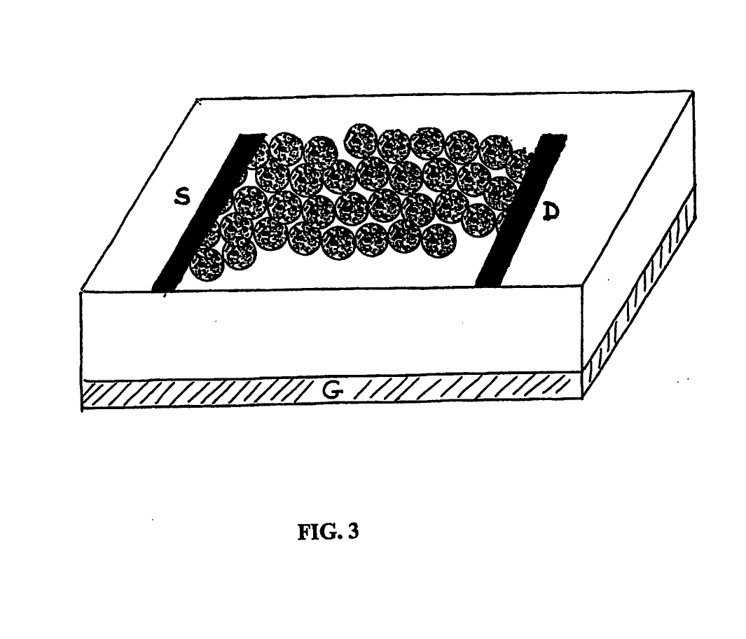

Such functionalized nano-structured material is shown in FIG. 2. The resistance of the nano-structured sensor material is monitored and the resistance value, with the biomaterial immobilized on the surface can be measured. The target molecules attach specifically to sensor attached biochemical receptor, this attachment resulting in a change of the charges at the surface layer of the nano-structured material. This change therefore leads to the change of the resistance of the device, or other electronic characteristics associated with the motion of the electrons across the device. Such change has been established in case of chemical species, and forms the basis of chemical sensor application of these materials. With the resistance monitored during the application of a bioassay, the change of the electronic signal leads to the detection of the conformational change, such as duplex formation or protein attachment to the immobilized biomaterial.

While the device is preferably used in a bioassay environment, but a simple construction can be made to detect biomaterials which exists in a gaseous or air environment (e.g. to sense biohazards such as smallpox and anthrax etc., toxic gases such as VX, satin, etc). In this case a device will typically consist of the elements comprising: an air flow system which takes samples of the gaseous or air environment; a mechanism which allows the sampled gas or air to dissolved in a bioassay; and a mechanism, such as used in a “lab on a chip” arrangement which flows the bioassay over the detector.

Illustrative Nanoparticle Network Fabrication

In typical embodiments of the invention, nanotube networks will initially be fabricated by direct deposition of single walled nanotubes on a silicon surface. While silicon is a typical surface, artisans understand that deposition to other surfaces is also contemplated. The networks can be used to fabricate transistor devices, with transconductances close to that obtained for an array of nanotubes with conducting and semiconducting nanotubes involved.

A typical example of the fabrication of such networks involves nanotube films. We have developed a method of laying down nanotube films employing porous alumina membranes as filters and measurement substrates. Such alumina membranes are an ideal substrate as they can be made optically flat and are easily characterized as a background material. The method has a number of important advantages over simple air drying of a liquid suspension for carbon nanotubes. The weak residual interaction between nanotubes in solution results in large flocculation effects (clumping) as the suspension dries. Air-drying of such a suspension on sapphire results in totally unsuitable results, where nanotubes form 0.1-mm ‘piles’ upon drying. Filtration through an inert substrate such as alumina allows liquid to be removed before large-scale structures form in the suspension. In this method, nanotubes are ultrasonically dispersed in spectroscopic grade dichlorobenzene or xylene. Then this suspension is deposited onto vacuum pumped alumina membranes (0.2 micron pore size) where the liquid can be removed on a time scale short enough to not allow flocculation to occur. In addition, we have recently developed a method whereby these deposited films can be subsequently floated on top of a water/isopropanol solution and then redeposited on a arbitrary substrate (silicon or sapphire). This method resembles the Langmuir-Blodgett film deposition that is used to create thin organic monolayer films.

The films consist of an interconnected network of bundles of single wall nanotubes, with a typical tubule diameter of 10 nm—comparable to a typical multiwall nanotube, one which significant protein attachment was found (see below).

Illustrative Electronic, Source-Drain Contact Fabrication

In typical embodiments of the invention, the sensor architecture includes a nanotube network with two contact electrodes attached, and combined with a simple fluidic cell. 1000 Angstroms of titanium is deposited through a mask, providing electrodes on the coverslip and film. Wires are then attached directly to the titanium which is in contact with the network using silver epoxy.

Buffers

Due to the finite conductivity of the buffer the device in a buffer environment can be represented as two parallel conducting channels, with both channels, in principle changing during the experiment.

The resistance of a SWNT network strongly depends on the density of the network. The objective is then to maximize to buffer's resistance while minimizing the network's resistance, but keeping in mind that the buffer's resistance has to be large enough (i.e. have enough salt) so that the protein finds itself in a biological environment.

Protein solutions where the protein concentration and buffer concentration spanned 6 orders of magnitude each were used with solutions of varying molecular weight and protein size.

In the context of the embodiments of the invention disclosed herein, artisans will understand that the term “nanoparticle” includes bulk nanoparticles, such as oxide nanoparticles, cocoons, nanowires, nanofibres, nanotubes, bundles of nanotubes, fullerenes and the like.

In the context of the embodiments of the invention disclosed herein, artisans will understand that the term “network” comprises a collection of nanoparticles as defined above, providing a conduction path between two electrodes. The conducting path dominantly includes the nanoparticles in dose proximity to each other, with the current flowing from one nanoparticle to the other, to the next, etc. In certain embodiments of the invention, networks are typical instead of a film due to their ability to: (1) provide a higher surface area; (2) conducting part more sensitive to environment and to attached/detected biomolecules; and (3) exhibit a size compatibility with proteins. In addition, a network, in contrast to individual nanoscale interconnects provides robustness, fault tolerance, and reproducibility while preserving sensitivity. Networks are distinguishable from a porous material.

In the context of the embodiments of the invention disclosed herein, artisans will understand that the term “semiconducting” simply means providing conduction. For example in embodiments of the invention, a network can include both semiconducting and metallic nanoparticles.

Embodiments of the invention disclosed herein can include a recognition layer. Typically this includes a polymer with protein-repelling properties, such as PEG, and others known in the art. Optionally this layer has self-assembly properties. In one embodiment of the invention, this layer is a dense set of antibodies.

Embodiments of the invention disclosed herein include a substrate, preferably a silicon or a polymeric substrate. Alternative substrate materials such as glass, metal, plastic and the like are contemplated. Embodiments of the invention disclosed herein further include electronic interconnects, for example in resistor (source and drain) and/or transistor (source and drain together with gate) configuration.

Advantage Over Prior Art

Devices known in the art include electronic devices where the sensing element is a continuous film and electronic devices with one nanoparticle element such as a nanowire or a nanotube. The disclosed architecture of certain embodiments of the invention have several advantages over existing devices. A first such advantage is a simplicity in fabrication. In addition, there is no need for patterned catalyst, and may be for a structure where nanoparticles are present in one location and not present in others on the wafer. Yet another advantage is a large surface area available for immobilization. Yet another advantage is a selected quasi one-dimensional conduction path. Yet another advantage is a size compatibility with proteins allowing protein selective immobilization. Yet another advantage is that as many nanoparticles act as the conducting element, statistical averaging will occur, strongly reducing the signal variation from device to device. Yet another advantage is that the signal is a simple dc resistance (or dc voltage), eliminating the need for an elaborate drive and detection electronics. Yet another advantage is that by virtue of the large number of nanoparticles involved, the structure is also “defect tolerant”.

### EXAMPLES

### Example 1

### Electrical Detection of Specific Protein Binding Using Nanotube FET Devices

This Example appears in publication: Star et al., NANO LETTERS 3(4): 459-463 (2003). The example involves one nanotube as sensing element but the art can be equally well applied to a collection of nanoparticles, in particular to nanotube networks of to networks of nanofibres, or any other nanostructured material onto which the polymers mentioned in the Example can be deposited.

In this example we used nanoscale field effect transistor devices with carbon nanotubes as the conducting channel to detect protein binding. A PEI/PEG polymer coating layer has been employed to avoid nonspecific binding, with attachment of biotin to the layer for specific molecular recognition. Biotin-streptavidin binding has been detected by changes in the device characteristic. Nonspecific binding was observed in devices without the polymer coating, while no binding was found for polymer-coated but not biotinylated devices. Streptavidin, in which the biotin-binding sites were blocked by reaction with excess biotin, produced essentially no change in device characteristic of the biotinylated polymer-coated devices.

Current biological sensing techniques commonly rely on optical detection principles that are inherently complex, requiring multiple steps between the actual engagement of the analyte and the generation of a signal, multiple reagents, preparative steps, signal amplification, complex data analysis, and relatively large sample size. The techniques are highly sensitive and specific but more difficult to miniaturize. Electronic detection techniques may offer an alternative, but their potential has not yet been explored fully. Field effect transistors (FETs) fabricated using semiconducting single wall carbon nanotubes (nanotube FETs, NTFETs) have been extensively studied (1,2). Such devices have been found to be sensitive to various gases, such as oxygen and ammonia, and thus can operate as sensitive chemical sensors. The mechanism responsible for the change of device characteristic is thought to be a charge-transfer reaction between the analytes and the nanotube. NTFET devices (3,4) together with devices based on nanowires (5), are also promising candidates for electronic detection of biological species. Various groups have examined the conformational compatibility—driven by size issues as well as hydrophobic effects—between proteins and carbon nanotubes using streptavidin, and found that the protein is able to crystallize in a helical conformation around multiwall carbon nanotubes (6). We have also shown (7) that functionalization of the nanotubes with carboxylic groups, thereby rendering them more hydrophilic, does not lead to protein attachment, thus opening up the avenues for specificity. Researchers have also made (3c,4) some attempts at functionalizing single-wall carbon nanotubes to make them biocompatible, capable of recognizing proteins by using noncovalent binding between a bifunctional molecule and the nanotube to anchor a bioreceptor molecule with a high degree of control and specificity.

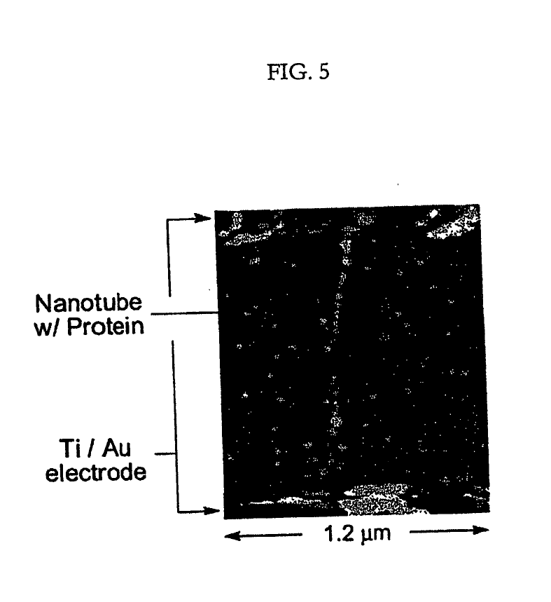

In this example we report taking these advances one step further, by using a sensor architecture that allows the detection of protein-receptor interactions (using biotin-streptavidin binding as an example) and, at the same time, reduces or eliminates nonspecific protein binding. FIG. 5 schematically depicts a sensor architecture that uses a NTFET as a transducer; it is covered with a polymer coating that has hydrophilic properties and onto which biotin is attached. Polymer functionalization in this sensor architecture has several advantages. First, the polymer is used to attach molecular receptor molecules to the sidewalls of nanotubes. Several examples of covalent chemical attachment of biological molecules to nanotubes, including proteins and DNA, have been recently published (8). Covalent modification, however, has the disadvantage that it impairs physical properties of carbon nanotubes. For these reasons we have employed a supramolecular approach, namely, the noncovalent functionalization of carbon nanotubes by employing polymer coatings (9). Second, polymer coatings have been shown to modify the characteristics of nanotube FET devices, and thus the coating process can be readily monitored. In particular, coating NTFETs with polyethylene imine (PEI) polymer was found (10) to shift the device characteristic from p- to n-type, presumably due to the electron-donating ability of amine groups in the polymer. Third, the polymer coating could be used to prevent nonspecific binding of proteins. A variety of polymer coatings and self-assembled monolayers have been used to prevent binding of undesired species on surfaces for biosensor and biomedical device applications (11). Among the various available polymers for coating, poly(ethylene glycol) (PEG) is one of the most effective and widely used. This layer, due to its hydrophilicity, reduces the affinity of nanotubes toward protein binding.

We have chosen the biotin-streptavidin binding to demonstrate the effectiveness of the device architecture. This binding serves as a model system for protein interactions (12) has been extensively studied, and the binding is well understood. In our procedure, after incubation, the device was washed and dried, and the device characteristics were examined after drying. While we have explored the device response in a buffer, our objective here is to examine the changes of the device characteristic, brought about by the different chemical and biological modifications on the electronic response, such direct correspondence being somewhat obscured in a buffer environment (13).

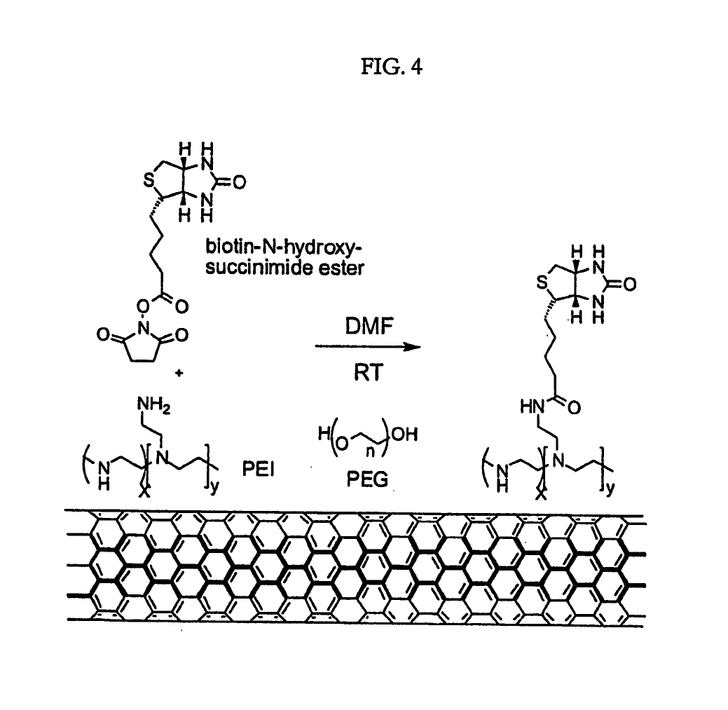

FET devices with nanotubes as the conducting channel were fabricated using nanotubes grown by chemical vapor deposition (CVD) on 200 nm of silicon dioxide on doped silicon from iron nanoparticles with methane/hydrogen gas mixture at 900° C.; electrical leads were patterned on top of the nanotubes from titanium films 35 nm thick capped with gold layers 5 nm thick, with a gap of 0.75 Ìm between source and drain. Multiple nanotubes connected the source and drain electrodes, with the individual tubes varying from metallic to semiconducting (14). Consequently, a range of device modulations (expressed as the ratio of the “on” to the “off” source-drain current, measured at −10 V and +10 V gate voltage, respectively) were observed. The devices displayed p-type transistor behavior, as has also been observed by others (1,2). In this example we have examined the dependence of the source-drain current, Isd, as function of the gate voltage Vg, Isd(Vg), measured from +10 V to −10 V, and we refer to this response as the “device characteristic”. After conducting initial electrical measurements to establish the device characteristic, the substrates were submerged in a 10 wt % solution of poly(ethylene imine) (PEI, average molecular weight _{—}25 000, Aldrich) and poly(ethylene glycol) (PEG, average molecular weight 10 000, Aldrich) in water overnight, followed by thorough rinsing with water. Commercial polyethyleneimine (PEI) was used; this form is highly branched, has a molecular weight of about 25 000, and contains about 500 monomer residues. About 25% of the amino groups of PEI are primary with about 50% secondary, and 25% tertiary. A thin layer (<10 nm) of polymer material coated the devices, as observed by atomic force microscopy. The polymer-coated devices were biotinylated by submerging them in a 15 mM DMF solution of biotin-N-hydroxysuccinimide ester (Sigma) at room temperature. This compound readily reacts with primary mines in PEI under ambient conditions, leading to changes of the device characteristic as will be discussed below. After soaking overnight, devices were removed, rinsed with DMF and deionized water, blown dry in nitrogen flow, and dried in a vacuum. FIG. 4 depicts the scheme by which biotin was attached to the polymer coating. The biotinylated polymer-coated devices were exposed to the 2.5 ÌM solution of streptavidin 15 in 0.01 M phosphate buffered saline (pH) 7.2, Sigma) at room temperature for 15 min. Subsequently, the devices were thoroughly rinsed with deionized water and blown dry with nitrogen. Several control experiments have also been performed in order to demonstrate the effectiveness of the polymer layer in the prevention of nonspecific binding.

An atomic force microscope (AFM) image of one of the devices after exposure to streptavidin labeled with gold nanoparticles is shown in FIG. 5. Light dots represent gold nanoparticles (10 nm), and thus indicate the presence of streptavidin. Based on the image, we conclude that streptavidin is effectively attached to the nanotubes, due to the strong adsorption of the PEI polymer to the sidewalls of the nanotubes, which was biotinylated after deposition. With a nanotube length of 800 nm and a gold sphere diameter of 10 nm, it is expected that, upon full coating, there are approximately 80 streptavidin molecules in direct interaction with the nanotube conducting channel. (This assumes that, on the average, one streptavidin molecule per gold nanoparticle is attached to the nanotube.)

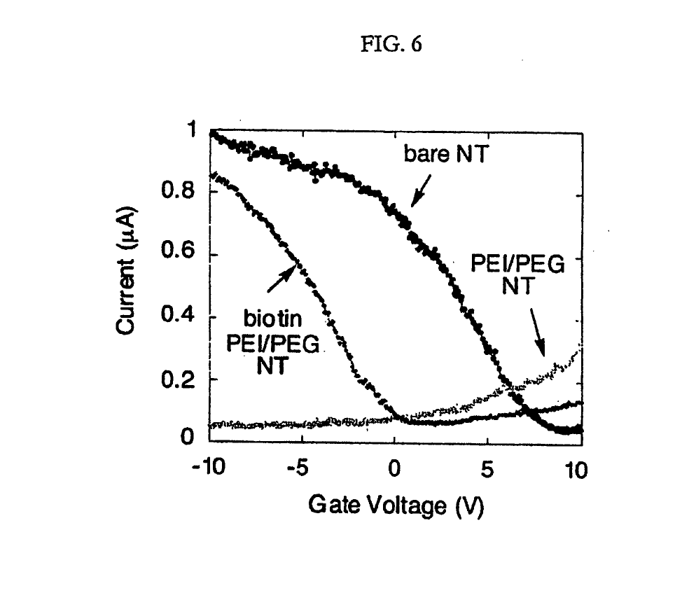

Next we discuss the change of the device characteristic in response to the steps we have taken. The device characteristic before chemical modification is p-type, in an ambient environment, presumably due to exposure to oxygen (16). Coating the device with the mixture of PEI and PEG polymers results in an n-type device characteristic (FIG. 6). This effect, which has been observed (10) before, probably results from the electron donating property of the NH2 groups of the polymer. The electronic characteristic of the device after 18 h of biotinylation reaction is also depicted in FIG. 6. Attachment of biotin is through covalent binding to the primary NH2 group, thereby reducing the overall electron donating function of PEI and leading to a device characteristic that is consistent with removal of electrons from the device. As only the primary NH2 sites are involved in binding to biotin, the p-type conductance observed before coating is not fully recovered (17).

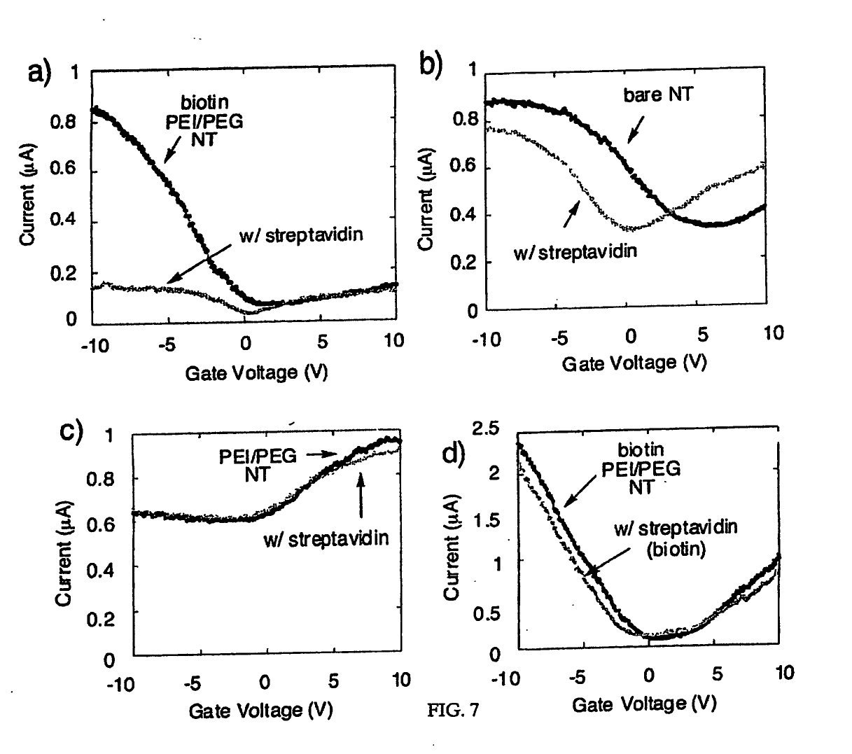

The effect of exposing the biotinylated polymer-coated device to a streptavidin solution and the control experiments (conducted on different devices) are shown in FIG. 7. FIG. 7a shows a striking loss of source-drain current for negative gate voltages after exposure to streptavidin and consequent streptavidin-biotin binding with little evidence for the shift of the device characteristic toward negative or positive gate voltage. Several control experiments were performed to demonstrate the effectiveness of the device architecture in avoiding false positives and in detecting specific protein binding. First, we have exposed the uncoated NTFET device to streptavidin and have found a change of the device characteristic, as shown in FIG. 7b, indicating attachment of streptavidin to the device. Note, however, that in this case the primary effect is the shift of the device characteristic toward negative gate voltage. In contrast, when the device was polymer-coated, but not biotinylated, no changes occurred upon exposure to streptavidin FIG. 7c). This suggests the effectiveness of the polymer coating in preventing direct, nonspecific interaction of streptavidin with the nanotube. Finally, addition of a streptavidin in which the biotin-binding sites were blocked by complexation with excess biotin produced essentially no change in device characteristic of the biotinylated polymer-coated device (FIG. 7d).

Several conclusions on the effect of biomolecules on the device electronics can be drawn. First, exposing the bare, uncoated device to streptavidin leads to the shift of the transconductance toward negative gate voltages, thereby rendering the device less p-type, with little reduction in the magnitude of the transconductance. This indicates that the primary effect of the nanotube-streptavidin binding is a charge-transfer reaction with streptavidin donating electrons to the nanotube (16). Biotin-streptavidin binding has a different effect; in this case the Isd is reduced. Without being bound by a specific theory, we suggest that upon streptavidin-biotin binding, geometric deformations occur, leading to scattering sites on the nanotube, and thus to reduced conductance. At the same time the device characteristic is modified only for negative gate voltages (see FIG. 7a), leaving the transconductance in the positive gate voltage region unaffected. We have observed (17) similar features in devices to which charge carriers were deposited, and we have argued that the observation is due to localization (delocalization) of positively (negatively) charged ionic entities by a negatively (positively) charged surface. Such a mechanism may also be effective here, and the mechanism may open the way for electronic modification of bioreactions.

With improvements in NTFET devices, they may also be rendered sensitive enough that single protein detection and monitoring can be achieved. As can be inferred from FIG. 7a, the total change in transconductance exceeds the noise level by a factor of 10. According to an AFM image of the device (FIG. 5), there are about 100 gold nanoparticles, and approximately 100 protein molecules (assuming one protein per gold nanoparticle binding to the tube) in close proximity to the carbon nanotube. Combining these two numbers, our current detection level can be estimated to be of the order of 10 streptavidin molecules. Similar detection sensitivity can be inferred from experiments we have conducted on uncoated nanotubes incubated with streptavidin (FIG. 7b). This is in contrast to the relatively modest change observed in devices where the active element is a nanowire 5—a channel with a substantially larger cross section.

Electronic sensing using devices with nanotubes as the conducting channel offers several advantages. Such sensors are small and fast, and the active detection area is sized for individual proteins or viruses. These sensors are extremely sensitive, as all the current passes through the detection point. Most importantly, at a later stage the devices can be made specific to individual molecules; potentially their response to different species can be varied in a controlled way using chemical and biological functionalization. These concepts could conceivably be extended at a later stage to include cell-based electronic sensing (measuring the electronic response of living systems) and to using nanoscale devices for in-vivo applications (studying cell physiology, medical screening and diagnosis). The devices can be turned into devices where, by applying a voltage between elements of the sensor, surface charges can be created on the sensing element where the bio-molecules are immobilized. Such surface charges will interact with the charged biomolecules affecting biological function.

### REFERENCES

(1) Bachtold, A.; Hadley, P.; Nakanishi, T.; Dekker, C. Science 2001, 294, 1317-1320. (2) Martel, R; Schmidt, T.; Shea, H. R.; Hertel, T.; Avouris, Ph. Appl. Phys. Lett. 1998, 73, 2447. (3) (a) Collins, P. G.; Bradley, K.; Ishigami M.; Zettl, A. Science 2000, 287, 1801. (b) Kong, J.; Franklin, N. R.; Zhou, C.; Chapline, M. G.; Peng, S.; Cho, K.; Dai, H. Science 2000, 287, 622. (c) Chen, R. J.; Zhang, Y.; Wang, D.; Dai, H. J. Am. Chem. Soc. 2001, 123, 3838-3839. (4) Shim, M.; Kam, N. W. S.; Chen, R. J.; Li, Y.; Dai. H. Nano Lett. 2002, 2, 285-288. (5) Cui, Y.; Wei, Q.; Park, H.; Lieber, C. M. Science 2001, 293, 1289-1292. (6) Balavoine, F.; Schultz, P.; Richard, C.; Mallouh, V.; Ebbesen, T. W.; Mioskowski, C. Angew. Chem., Int. Ed. Engl. 1999, 38, 1912-1915. (7) Gruner, G.; Gabriel J.-C.; Zocchi, G., unpublished. (8) (a) Baker, S. E.; Cai, W.; Lasseter, T. L.; Weidkamp, K. P.; Hamers, R. J. Nano Lett. 2002, 2, 1413-1417. (b) Huang, W.; Taylor, S.; Fu, K.; Lin, Y.; Zhang, D.; Hanks, T. W.; Rao, A. M.; Sun, Y.-P. Nano Lett. 2002, 2, 311-314. (9) (a) Star, A.; Stoddart, J. F.; Steuerman, D.; Diehl, M.; Boukai, A.; Wong, E. W.; Yang, X.; Chung, S. W.; Choi, H.; Heath, J. R. Angew. Chem., Int. Ed. 2001, 40, 1721-1725. (b) O'Connell, M. J.; Boul, P.; Ericson, L. M.; Huffman, C.; Wang, Y. H.; Haroz, E.; Kuper, C.; Tour, J.; Ausman, K. D.; Smalley, R. E. Chem. Phys. Lett. 2001, 342, 265-271. (c) Star, A.; Steuerman, D. W.; Heath, J. R.; Stoddart, J. F. Angew. Chem., Int. Ed. 2002, 41, 2508-2512. (10) Shim, M.; Javey, A.; Kam, N. W. S.; Dai, H. J. J. Am. Chem. Soc. 2001, 123, 11512-11513. (11) Ostuni E.; Chapman, R. G.; Holmlin, R. E.; Takayama, S.; Whitesides, G. M. Langmuir 2001, 17, 5605-5620, and references therein. (12) (a) Miyamoto, S.; Kollman, P. A. Proteins Struct. Funct. Genet. 1993, 16, 226-245. (b) Vajda, S.; Weng, Z.; Rosenfeld, R.; DeLisi, C. Biochemistry 1994, 33, 13977-13988. (13) Star, A.; Han, T. R.; Gabriel J.-C.; Bradley, K.; Gruner, G. “Electronic Detection in Liquids Using Nanotube FET Devices”, submitted for publication. (14) NTFETs were fabricated using nanotubes grown by chemical vapor deposition, directly on 4″ silicon wafers (with 200 nm films of thermal SiO2) using a home-built apparatus. In a typical experiment, the wafer is covered with patterned photoresist and is spin coated with growth promoter containing nanoparticles of iron encased within a mesoporous material [(a) Li, W. Z.; Xie, S. S.; Qian, L. X.; Chang, B. H.; Zou, B. S.; Zhou, W. Y.; Zhao, R. A.; Wang G. Science 1996, 274, 1701-1703, (b) Pan, Z. W.; Xie, S. S.; Chang, B. H.; Wang, C. Y.; Lu, L.; Liu, W.; Zhou, W. Y.; Li, W. Z.; Qian, L. X. Nature 1998, 394, 631-632]. After liftoff in acetone, small patterned areas of the growth promoter are left on the wafer. The wafer is then introduced in a 5 in. tubular oven and treated at 900° C. in a methane and hydrogen flow for 15 min, allowing for the growth of SWCNT of 5-10 Ìm long and 1.5 to 3 nm in diameter. The hydrogen helps to prevent the deposition of amorphous carbon around the nanotubes as well as on the surface of the silicon [Ivanov V.; Nagy, J. B.; Lambin, Ph.; Lucas, A.; Zhang, X. B.; Zhang, X. F.; Bernaerts, D.; Van Tendeloo, G.; Amelinckx, S.; Van Landuyt, J. Chem. Phys. Lett. 1994, 223, 329-335]. Standard optical lithography and metal deposition are used to form the metal contacts on top of the grown nanotubes. A fair proportion of the tens of thousands of devices made on each wafer are p-type FETs with a modulation of 1, indicating that only semiconducting nanotubes are present. The devices used in this study were selected from among those. An AFM image of a typical device is presented in Supporting Information. (15) Streptavidin is labeled with gold nanoparticles for the purpose of AFM imaging. Streptavidin (from Streptomyces aVidinii, Sigma Chemicals) without gold labeling had similar effect on the device characteristic but could not be detected by AFM. (16) (a) Jhi, S.-H.; Louie, S. G.; Cohen, M. L. Phys. ReV. Lett. 2000, 85, 1710-1713. (b) Ulbricht, H.; Moos, G.; Hertel, T. Phys. ReV. B 2002, 66, 075404. (17) The progress of the on-chip biotinylation reaction can be monitored by measuring Isd(Vg). Biotin-N-hydroxysuccinimide ester reacts readily with primary amines in PEI under ambient conditions, thus reducing the electron donating of PEI. However, after 1 h the yield of the reaction is only _{—}75%; several hours are required to complete the reaction. The device characteristic at different stages of the biotinylation reaction is presented in Supporting Information. (18) Wilchek, M.; Bayer, E. A. Methods Enzymol. 1990, 184, 49. (19) Cumings, J.; Star, A.; Gabriel, J.-C.; Bradley, K.; Gru\ddot{}ner, G. “Influence of Mobile Ions on Nanotube Based FET Devices”, submitted for publication.

### Example 2

The affinity of proteins to nanotubes was explored by using multiwall nanotubes and also a network of bundles of nanotubes, both the multiwall nanotubes and the bundles having a diameter similar to the diameter of the protein in question. Nanotubes (2 mg) dispersed in were incubated with BSA-dye conjugate (1 mg/ml, 1.25 microgram) overnight, were washed with PBS. Depletion of BSA from the solution was evaluated using UV absorption (580 nm) of fluorescent dye. Significant depletion was observed, however the amount of attached protein per nanotube was be evaluated as the surface area of the bundles was not measured. SEM images indicated significant coverage, as observed by other groups.

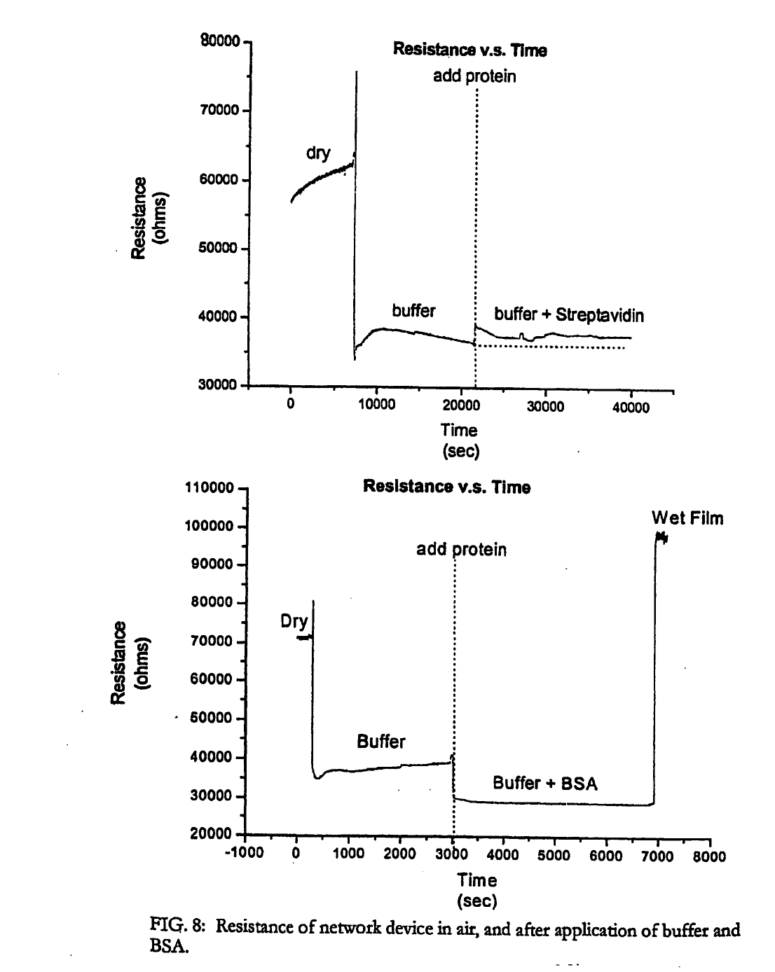

When adding proteins (BSA) the resistance drops, as illustrated on FIG. 8. This is because the BSA solution applied is 4 times more conductive than the buffer solution. When calculating what value of resistance the film has (considering the network as a resistance in parallel with buffer) it is roughly 30% higher than the value of the resistance in buffer without the protein added. This can also be seen at the end of the data run when the fluid is removed from the film. The resistance increases above what the dry value of resistance of the film.

The data were analyzed by solving a set of linear equations which related the networks resistance at each point to the film's resistance when the film is initially submerged in the fluid (during “Buffer” section on graph). This is done using the relationship between the conductivity of the buffer and the conductivity of the protein solution.

$$- \frac{\frac{1}{Rf\_prot} + \frac{1}{Rtot\_prot}}{- \frac{1}{Rf\_buf} + \frac{1}{Rtot\_buf}} = \frac{Lprot}{Lbuf}$$

Where the left-hand-side of the equation is just Rbuf/Rprot, and
Lbuf=conductivity of buffer solution
Lprot=conductivity of protein solution
Rtot_buf=total resistance measured while submerged in buffer (see “Buffer” in figure)
Rtot_prot=total resistance measured while submerged in protein (see “Protein” in figure)
Rf_buf=Film's Resistance in Buffer (this is the initial “guess”, based on “Dry”)
Rf_prot=Film's Resistance in Protein (solve for in equation above)

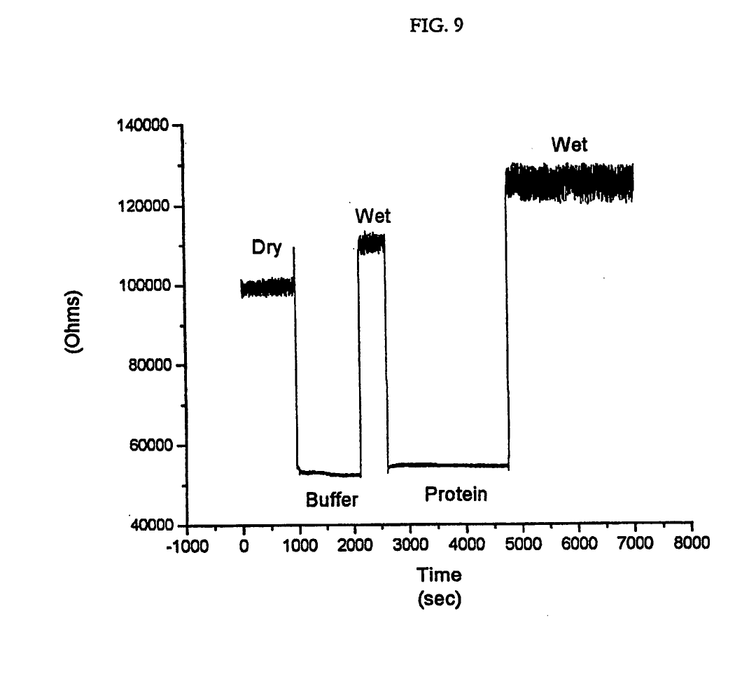

A simpler analysis can be made where the values of the film's resistance are assumed to correspond to the values measured on the “wet” section of the graph (see FIG. 9). This however leads to inconsistencies when compared to the measurements of the conductivities of the buffer/protein solutions. Therefore, the more detailed analysis is typical, and is described below.

Data:

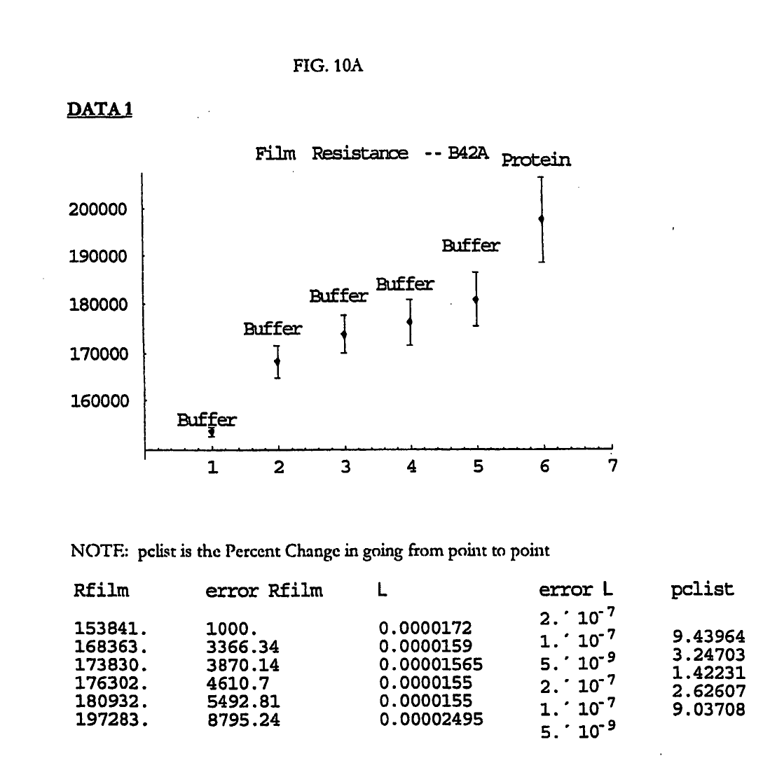

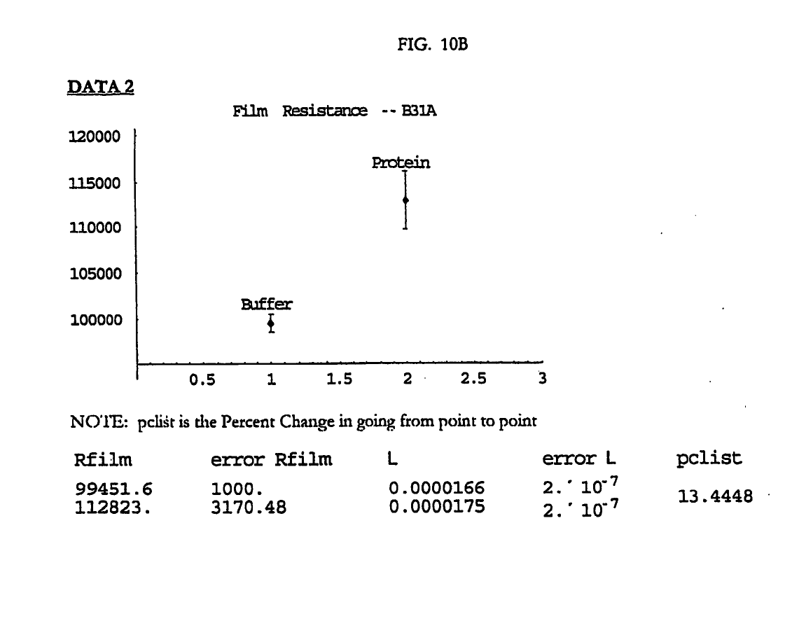

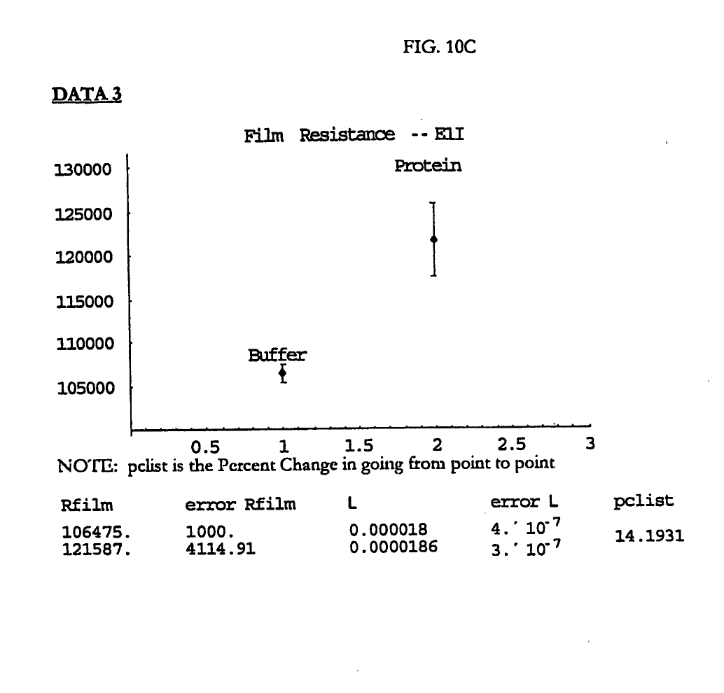

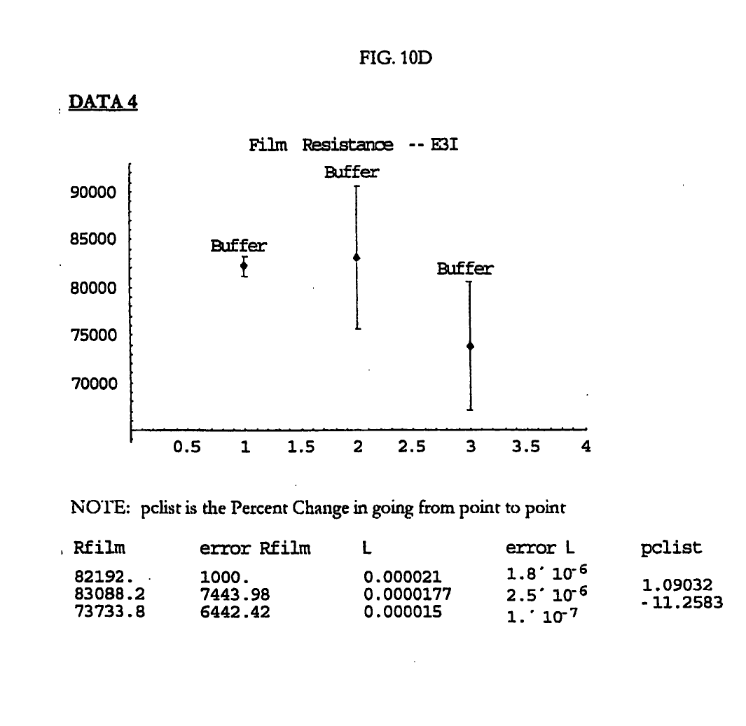

The data below show the film's resistance. There are 2 different cases:

| | |
|---|---|
| 1) Add Buffer, Add Protein | (3 data sets, FIGS. 10A-10C) |
| 2) Add Buffer, Add Buffer | (1 data set, FIG. 10D) |

2 different protein concentrations were used. For Data 1 (FIG. 10A), BSA of approximately 1.3 micro-molar (micro Moles/Liter) was used. For Data 2 and 3 (FIGS. 10B and 10C), BSA of approximately 13 micro-molar was used. The buffer used was PBS of approximately 5 micro-molar concentration.

### CONCLUSION

In all the data sets where protein is added, the resistance of the film increases by ˜10%. In the first data set the network was washed with buffer many times before adding the proteins. In this data set (FIG. 10 A) the film's resistance increases by 9% when the buffer is added, and then much less each time more buffer is added (˜3%). This latter increase is most likely due to tubes being washed away, and is seen to diminish as we make more replacements of the buffer. When the protein solution is added, there is a 9% jump in the film's resistance. This is a strong case for having a noticeable change upon addition of proteins. Taking error bars into account, there is still a ˜1.1% increase in the networks resistance.

In conclusion, nanotube networks offer appropriate alternatives to detection of proteins. Such networks could also be functionalized for binding specificity, using strategies similar to those explored using nanotube based FET devices discussed in Example 1.

### Example 3

### Analyte Sensing Under Multiple Sensing Conditions

A. Illustrative Experiments Using a Typical Sensing Condition. The Influence of Constant Gate Voltage on Binding.

FIG. 11 shows the electric field dependence of the change of the device characteristics upon incubation with streptavidin. Electric fields will also have different influence the binding of other biomolecules such as antibodies and antigens and complementary polynucleotide sequences. Consequently, in one embodiment of the invention, analyte identification is possible by: (1) measuring the change of the device characteristics without application of an electric field, and repeating the experiments with an applied electric field and comparing with the result without an electric field.

In addition, local electric field will influence the interaction between different biomolecules, with a typical example of a DNA duplex. A sufficiently large electric field can lead to the “melting” of the duplex, and measuring the field where this occurs can lead to the identification of the base pair sequence.

B. Illustrative Experiments Using a Typical Sensing Condition. The Influence of PH on Binding.

This part shows how change of the external parameter, pH in this case, leads to distinguishing between two different proteins: Streptavidin (SA) and Bovine Serum Albumin (BSA).

First, the pH response of bare device in 150 mM PBS buffer was measured, using the technique described in Part 1 above. The pH had been changed from buffer's original value of 7.4 down to 3 by slow adding hydrochloric acid (HCl) with the same ionic strength as buffer. After that, device was fully covered with proteins by injecting 100 nM BSA or SA to the buffer solution with the following rinse. And finally, the pH response of protein covered device was measured again.

From the disclosure provided in FIGS. 12A and 12B, it can be concluded that Streptavidin introduces insignificant modifications to pH response of the device. On the other hand, BSA noticeably changes both Tilt and Shift characteristics. As shown in FIG. 12C, if the response of bare device is taken as the baseline, then the relative to baseline Shift clearly distinguish between two proteins. The difference becomes mote significant at lower pH, which might be due to the fact that BSA changes its conformational state from normal to so called fast one (N-F transition) at acetic conditions (see, e.g. Khan, M. Y. Biochem. J. 1986, 236, 307).

C. Typical Electrochemical Setup and Measurement Techniques.

Transducer: As an example of an electronic circuit, containing one dimensional material as conducting channel, the carbon nanotube network field-effect transistor was chosen. The network of carbon nanotubes was grown by chemical vapor deposition process from iron nanoparticles placed on silicon oxide surface producing nanotubes having 1-3 nm in diameter. Source-drain electrical leads were formed by optical lithography (35 nm titanium covered with 5 nm gold). The most often geometry used was the one where 2 micron thick source and drain contact lines were separated by 10 microns and had 200 microns in length, yielding 2000 square microns of sensing area.

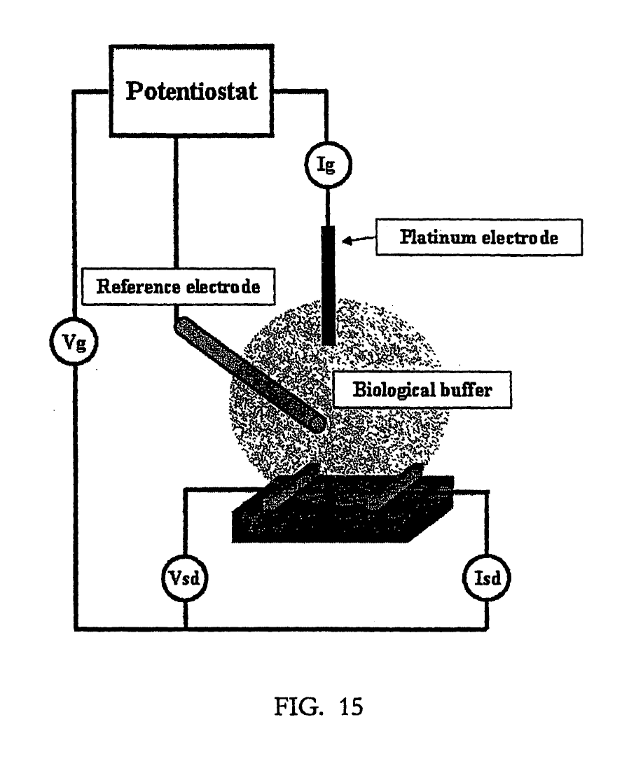

Experimental arrangement: The chip, containing the device, was encapsulated with chemically resistive epoxy in such way that only carbon nanotube network and source-drain leads as well as epoxy itself were exposed to aqueous solution. The teflon cap was hermetically sealed on top of the device allowing addition or removal biological buffer in the range from 1 to 50 ml. The cover of the cup had several holes with rubber seals for electrochemical electrodes and biological material injection/withdrawal lines. The PC computer with data acquisition card (DAQ) controlled applied source drain bias (Vsd), gate potential (Vg), recording at the same time source-drain current (Isd) and parasitic leakage current through gate electrode (Ig). The home-build potentiostat was employed to fix liquid potential using either saturated calomel (SCE) or silver/silver chloride (4M Ag/AgCl) low flow rate single junction reference electrodes. The schematic setup is shown in FIG. 15.

Measurements: A device can interact with analyte directly (protein sticking to nanotubes) or indirectly (nanotubes functionalized with antibodies bind antigens), but in any case, the conduction through nanotube network can be affected in two ways. Firstly, carrier mobility of the nanotube channels can change. Also, there can be a charge transfer between substances and nanotubes that changes carrier concentration in the semiconducting nanotubes. The simplest way to monitor real-time chemical or biological reactions is to measure the conduction through source-drain contacts as a function of time at a fixed liquid potential, which is done in the most recent papers (see, e.g. Besteman et al. Nano Lett. 2003, 3, 727; Chen et al. J. Am. Chem. Soc. 2004, 126, 1563; and Chen et al. Proc. Natl. Acad. Sci. U.S.A. 2003, 100, 4984), i.e., to exploit the device as a resistor. Nevertheless, since conduction is proportional both to carrier mobility and concentration, one extracted parameter cannot fully elucidate which of two (or both) effects have happened.

A more informative way to analyze processes going on in a nanotube field transistor is to record transfer characteristics (TC, or I_{sd}-V_{g}, or just IVg). The I_{sd}-V_{g} curve is obtained by sweeping gate voltage, while keeping source-drain potential constant Acquired I_{sd}-V_{g} curve is recorded to computer memory (typically 64 points per curve) for the future analysis (see, e.g. Bradley et al. Nano Lett. 2004, 4, 253; and Bradley et al. Phys. Rev. Lett. 2003, 91, 218301). This technique enables tracing relative changes both in carrier mobility (tilt of I_{sd}-V_{g} curve) and carrier concentration (shift of I_{sd}-V_{g} curve). The range of accessible gate voltages is limited by the presence of electrochemical reactions (faradic currents) between nanotubes and the electrolyte, which can interfere with currents through the network itself, that is why it is important to monitor gate current I_{g} along with I_{sd}. Usually, there are no faradic currents in the system if the gate voltage is swept at −0.2V+/−0.3V versus saturated calomel electrode (SCE).

In summary, a typical experiment is conducted in the following way.
Source and drained contacts are biased with small V_{sd} voltage from 10 to 30 mV.
Gate is swept with frequency around/below 1 Hz.
Gate offset is −0.2 V, gate amplitude is 0.3 V.
Gate output channel is connected to a potentiostat, which uses SCE as a reference, and platinum wire as an indicating (working) electrode.
Currents through the network and working electrode (gate current) are measured simultaneously. The final output of one measurement is I_{sd}-V_{g} and I_{g}-V_{g} curves.
Usually 16,384 data points per I_{sd}-V_{g} curve are collected, and then averaged to 64 points.
All data rows are stored in computer memory and have the following format: time at which I_{sd}-V_{g} curve is taken, V_{sd} bias, gate frequency, V_{g} offset, V_{g} amplitude, average I_{sd} noise, temperature, pH, 64 dusters consisting of 3 points: (V_{g}, I_{sd}, I_{g}).

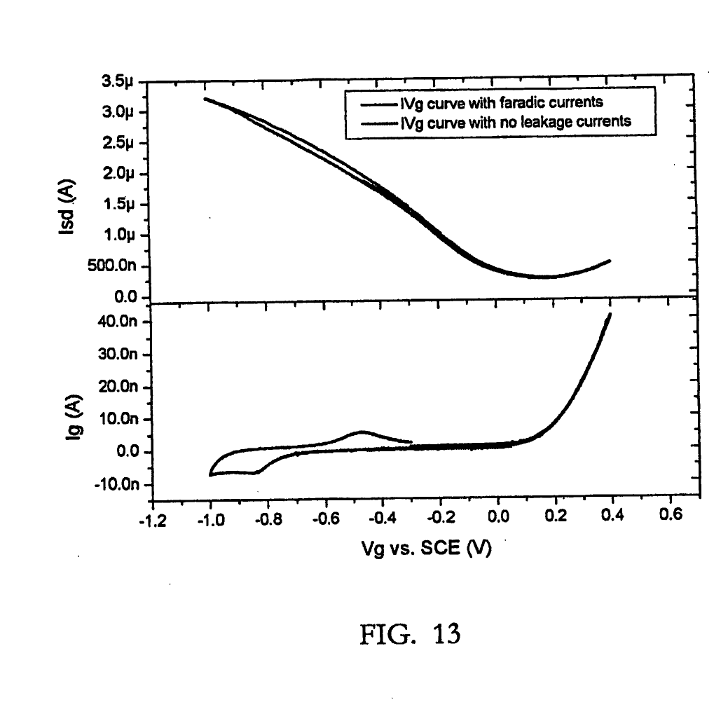

As an example, FIG. 13 shows two I_{sd}-V_{g} curves for the same device. In the case of where parasitic currents are noticeable, the hysteresis is larger, which makes data interpretation more difficult. Also, in general, it was noted that devices operating in the region with faradic currents are less stable than devices with no leakage currents present.

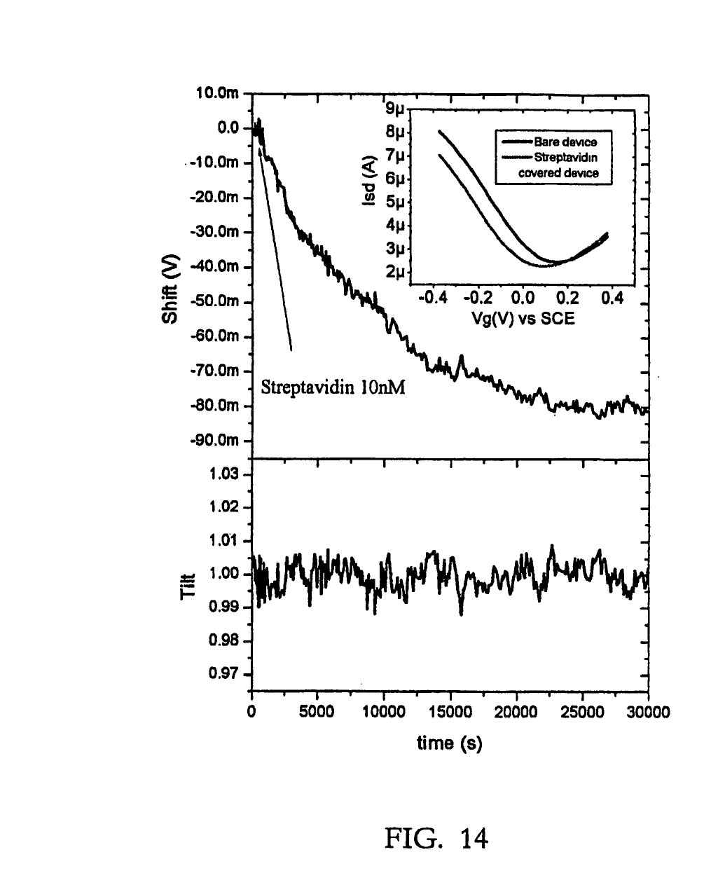

Example of technique application: Another example demonstrates how the technique can be used for real time monitoring of non-specific binding of streptavidin to carbon nanotubes. As it can be seen from the FIG. 14, streptavidin influences the device by electron transfer that is why I_{sd}-V_{g} curve shifts to the left, i.e. concentration of holes, which are original charge carries, decreases. On the other hand, since the tilt remains stable, it can be concluded that streptavidin doesn't affect carrier mobility.

Throughout this application, various publications are referenced (articles such as Snow et al., Appl. Phys. Lett. 82(13): 2145-2147 (2003) and Chen et al., PNAS 100: 4984-4989 (2003); patents, patent applications etc.). The disclosures of these publications are hereby incorporated by reference herein in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1. A method of sensing binding of an analyte to a composition capable of binding the analyte comprising:
(a) exposing the analyte to a sensor comprising:
an electric circuit coupleable to a device that monitors alterations in an electrical property of the electric circuit;
a composition capable of binding the analyte coupled to the electric circuit; wherein an electrical property of the electric circuit is altered when the analyte binds the composition capable of binding the analyte;
(b) monitoring the sensor under a first sensing condition comprising a first temperature, a first pH, a first buffer composition or a first electric field;
(c) monitoring the sensor under a second sensing condition comprising a second temperature, a second pH, a second buffer composition or a second electric field;
wherein, a change in an electrical property under the first sensing condition as compared to the second sensing condition correlates to the binding of the analyte to the composition capable of binding the analyte; and
(d) sensing the binding of the analyte to the composition capable of binding the analyte by observing a change in the electrical property observed under the first sensing condition as compared to the second sensing condition.

2. The method of claim 1, wherein the first and second sensing conditions are a first and second electric field.

3. The method of claim 2, wherein the electric field is altered so as to alter the binding of the analyte to the composition capable of binding the analyte.

4. The method of claim 1, wherein the composition capable of binding the analyte comprises a polynucleotide.

5. The method of claim 1, wherein the analyte comprises a target polynucleotide and the composition capable of binding the analyte comprises a sensing polynucleotide having sequences complementary to the target polynucleotide.

6. The method of claim 5, wherein the change in the electrical property observed under the first sensing condition as compared to the second sensing condition is correlated to the degree of complementarity between the target polynucleotide and the sensing polynucleotide having sequences complementary to the test polynucleotide.

7. The method of claim 1, wherein the method uses a plurality of sensors to perform the method on a plurality of different analytes as part of a sensing array.

8. The method of claim 1, wherein the method uses a single sensor that comprises a plurality of compositions capable of binding a plurality of different analytes to sense a plurality of different analytes.

9. The method of claim 1, wherein the electrical property is current, voltage or capacitance.

10. An analyte sensor system comprising:
(a) an electric circuit coupleable to a device that monitors alterations in an electrical property of the electric circuit;
(b) a composition capable of binding the analyte coupled to the electric circuit; wherein an electrical property of the electric circuit is altered when the analyte binds the composition capable of binding the analyte;
(c) a fluid delivery element adapted to deliver a fluid that may contain an analyte to the sensor;
(d) a sensor conditioning element adapted to alter the condition under which the analyte is sensed so that the system is adapted to:
(i) monitor an electrical property of the sensor under a first sensing condition comprising a first temperature, a first pH, a first buffer composition or a first electric field; and subsequently
(ii) monitor an electrical property of the sensor under a second sensing condition comprising a second temperature, a second pH, a second buffer composition or a second electric field;
wherein, a change in an electrical property under the first sensing condition as compared to the second sensing condition correlates to the binding of the analyte to the composition capable of binding the analyte.

11. The system of claim 10, wherein the system is coupleable to a processor that compares the electric property observed under the first sensing condition and the electrical property observed under the second sensing condition.

12. The system of claim 10, wherein the processor uses an algorithm to provide a signal when the difference between the electric property observed under the first sensing condition and the electrical property observed under the second sensing condition meets a predefined parameter.

13. The system of claim 10, wherein the system comprises a resistor, a transistor or a capacitor.

14. The system of claim 10, wherein the fluid delivery element is adapted to circulate a fluid over the sensor.

15. The system of claim 10, wherein the fluid delivery element comprises a fluid pump, a fluid conduit or a pipette.

16. The system of claim 10, wherein the system is adapted to continuously monitor an electrical property of the electrical circuit under different sensing conditions.

17. The system of claim 10, wherein the analyte or the composition capable of binding the analyte is not labelled with a detectable marker.

18. The system of claim 10, wherein the sensor conditioning element is a heater, a mixer, a fluid conduit or a pipette.

19. The system of claim 10, wherein the sensor comprises an insulating layer that inhibits binding of an analyte to a portion of the sensor.

20. The system of claim 10, wherein the composition capable of binding the analyte comprises a polypeptide.

21. The system of claim 10, wherein the change in the electrical property observed under the first sensing condition as compared to the second sensing condition is correlated to affinity or avidity that the analyte has for the composition capable of binding the analyte.

22. The system of claim 10, wherein the analyte comprises an antigen and the composition capable of binding the analyte comprises an antibody that binds the antigen.

23. The system of claim 10, wherein the sensor does not comprise a nanostructured material.

24. The system of claim 10, wherein the system comprises:
(a) a plurality of sensors to perform the method on a plurality of different analytes as part of a sensing array; or
(b) a plurality of compositions capable of binding a plurality of different analytes to sense a plurality of different analytes.

25. The system of claim 10, wherein the electrical property is current, voltage or capacitance.
